SEQUENCE LISTING (30 sequences)

SEQ ID NO: 1 - protein, 5 aa, Artificial Sequence
  CCR2 binding peptide
TFLKC

SEQ ID NO: 2 - protein, 7 aa, Artificial Sequence
  CCR2 binding peptide
  MISC_FEATURE (1)..(1): Xaa in position 1 is absent, is glycine or represents an amino acid sequence selected from the group consisting of LG, YLG, and HYLG.
  MISC_FEATURE (7)..(7): Xaa in position 7 independently is absent, is methionine, or represents an amino acid sequence selected from the group consisting of MA, MAN, MANG, MANGF, MANGFV, MANGFVW, MANGFVWE, and MANGFVWEN
XTFLKCX

SEQ ID NO: 3 - protein, 7 aa, Artificial Sequence
  ECL1 (C) inverso; ECL1i
LGTFLKC

SEQ ID NO: 4 - protein, 11 aa, Artificial Sequence
  CCR2 binding peptide
HYLGTFLKCMA

SEQ ID NO: 5 - protein, 9 aa, Artificial Sequence
  CCR2 binding peptide
LGTFLKCMA

SEQ ID NO: 6 - protein, 9 aa, Artificial Sequence
  CCR2 binding peptide
HYLGTFLKC

SEQ ID NO: 7 - protein, 11 aa, Artificial Sequence
  CCR2 binding peptide
GTFLKCMANGF

SEQ ID NO: 8 - protein, 11 aa, Artificial Sequence
  CCR2 binding peptide
TFLKCMANGFV

SEQ ID NO: 9 - protein, 16 aa, Artificial Sequence
  CCR2 binding peptide
HYLGTFLKCMANGFVW

SEQ ID NO: 10 - protein, 7 aa, Artificial Sequence
  ECL1 (C)
CKLFTGL

SEQ ID NO: 11 - protein, 5 aa, Artificial Sequence
  ECL2 (N)
LFTKC

SEQ ID NO: 12 - protein, 5 aa, Artificial Sequence
  ECL2 (N) inverso
CKTFL

SEQ ID NO: 13 - protein, 7 aa, Artificial Sequence
  ECL3 (C)
HTLMRNL

SEQ ID NO: 14 - protein, 7 aa, Artificial Sequence
  ECL3 (C) inverso
LNRMLTH

SEQ ID NO: 15 - protein, 7 aa, Artificial Sequence
  ECL3 (N)
LNTFQEF

SEQ ID NO: 16 - protein, 7 aa, Artificial Sequence
  ECL3 (N) inverso
FEQFTNL

SEQ ID NO: 17 - protein, 4 aa, Artificial Sequence
  CCR2 binding peptide
TFLK

SEQ ID NO: 18 - protein, 6 aa, Artificial Sequence
  CCR2 binding peptide
  MISC_FEATURE (1)..(1): Xaa in position 1 is absent, is glycine or represents an amino acid sequence selected from the group consisting of AG, LG, YLG, and HYLG.
  MISC_FEATURE (6)..(6): Xaa in position 6 independently is absent or is alanine.
XTFLKX

SEQ ID NO: 19 - protein, 6 aa, Artificial Sequence
  CCR2 binding peptide
LGTFLK

SEQ ID NO: 20 - protein, 7 aa, Artificial Sequence
  CCR2 binding peptide
AGTFLKC

SEQ ID NO: 21 - protein, 7 aa, Artificial Sequence
  CCR2 binding peptide
LGTFLKA

SEQ ID NO: 22 - protein, 5 aa, Artificial Sequence
  CCR2 binding peptide
GTFLK

SEQ ID NO: 23 - protein, 7 aa, Artificial Sequence
  CCR2 binding peptide
AGTFLKA

SEQ ID NO: 24 - protein, 4 aa, Artificial Sequence
  CCR2 binding peptide
MANG

SEQ ID NO: 25 - protein, 5 aa, Artificial Sequence
  CCR2 binding peptide
MANGF

SEQ ID NO: 26 - protein, 6 aa, Artificial Sequence
  CCR2 binding peptide
MANGFV

SEQ ID NO: 27 - protein, 7 aa, Artificial Sequence
  CCR2 binding peptide
MANGFVW

SEQ ID NO: 28 - protein, 8 aa, Artificial Sequence
  CCR2 binding peptide
MANGFVWE

SEQ ID NO: 29 - protein, 9 aa, Artificial Sequence
  CCR2 binding peptide
MANGFVWEN

SEQ ID NO: 30 - protein, 4 aa, Artificial Sequence
  CCR2 binding peptide
HYLG

(19) United States
(12) Patent Application Publication
(10) Pub. No.: US 2023/0381349 A1
(43) Pub. Date: Nov. 30, 2023

(54) METHODS AND COMPOSITIONS FOR IMAGING AND TREATING CANCER

(71) Applicant: WASHINGTON UNIVERSITY, St. Louis, MO (US)

(72) Inventors: Yongjian Liu, St. Louis, MO (US); Xiaohui Zhang, St. Louis, MO (US); Kian-Huat Lim, St. Louis, MO (US)

(73) Assignee: Washington University, St. Louis, MO (US)

(21) Appl. No.: 18/248,191

(22) PCT Filed: Oct. 6, 2021
(86) PCT No.: PCT/US2021/053719

§ 371 Date: Apr. 6, 2023

Related U.S. Application Data
(60) Provisional application No. 63/088,062, filed on Oct. 6, 2020.

(51) Int. Cl. A61K 51/10; A61K 31/506; A61K 47/10; A61K 31/337; A61P 35/00; A61K 51/12

(52) U.S. Cl. CPC A61K 51/1033; A61K 31/506; A61K 47/10; A61K 31/337; A61P 35/00; A61K 51/1244

(57) ABSTRACT

The present disclosure reports a C-C chemokine type 2 receptor (CCR2) targeting mediated nanoimmunotherapy to treat cancer, such as pancreatic ductal adenocarcinoma (PDAC). Also disclosed herein is the use of a biodegradable copper nanocluster (CuNC) for enhanced loading of chemotherapeutic drug, such as Gemcitabine (Gem).

### CROSS-REFERENCE TO RELATED APPLICATIONS

This application claims priority from U.S. Provisional Application Ser. No. 63/088,062 filed on Oct. 6, 2020, which is incorporated herein by reference in its entirety.

### STATEMENT REGARDING FEDERALLY SPONSORED RESEARCH OR DEVELOPMENT

This invention was made with government support CA196510 awarded by the National Institutes of Health. The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention (file name “019463-WO_Sequence_Listing_ST25.txt” created Monday, Oct. 4, 2021; 6,382 bytes). The subject matter of the Sequence Listing is incorporated herein by reference in its entirety.

### FIELD OF THE INVENTION

The present disclosure generally relates to theranostics, cancer therapy, immunotherapy, and targeted imaging.

### SUMMARY OF THE INVENTION

Among the various aspects of the present disclosure is the provision of methods and compositions for imaging or treatment of cancer. An aspect of the present disclosure provides for a CCR2-targeting composition comprising: a CCR2 binding peptide comprising a TFLK sequence; a chemotherapeutic agent moiety; or a nanoparticle. In some embodiments, the CCR2-targeting composition is capable of binding to CCR2; the CCR2 binding peptide is no more than 200 amino acids in length. In some embodiments, the CCR2 binding peptide is conjugated to the nanoparticle by direct covalent bonding or with a linker. In some embodiments, the chemotherapeutic agent moiety is conjugated to the nanoparticle by direct covalent bonding or with a linker. In some embodiments, the CCR2-targeting composition is an anti-cancer composition, an imaging agent, or both. In some embodiments, the CCR2-targeting composition (e.g., CuNPs-ECL1i) has an average diameter of about 2.7±0.2 nm or has a diameter less than about 10 nm. In some embodiments, the nanoparticle is an ultrasmall nanocluster comprising ⁶⁴Cu. In some embodiments, the CCR2-targeting composition enables imaging or delivery of a therapy to a tumor (e.g., a CCR2-expressing tumor, pancreatic ductal adenocarcinoma (PDAC) tumor). In some embodiments, the nanoparticle is an ultrasmall copper nanocluster, the chemotherapeutic agent moiety is gemcitabine, or the CCR2 binding peptide is ECL1i. In some embodiments, the linker is PEG or a PEG derivative. In some embodiments, the linker is a protease cleavable linker, optionally a cathepsin B-sensitive dipeptide linker. Another aspect of the present disclosure provides for a pharmaceutical composition comprising the CCR2-targeting composition described herein and, optionally, an immunotherapeutic agent. In some embodiments, the CCR2-targeting composition is an anti-cancer composition, an imaging agent, or both. In some embodiments, the immunotherapeutic agent is at least one immune checkpoint inhibitor. In some embodiments, the immune checkpoint inhibitor is anti-CTLA or anti-PD-1 antibodies, or both. Yet another aspect of the present disclosure provides for a pharmaceutical composition comprising the CCR2-targeting composition described herein and, optionally, a second chemotherapeutic agent (e.g., paclitaxel, nab-paclitaxel). In some embodiments, the CCR2-targeting composition is an anti-cancer composition, an imaging agent, or both. Yet another aspect of the present disclosure provides for a method of treating and/or imaging a subject having cancer comprising: administering an effective amount of the CCR2-targeting composition described herein. In some embodiments, the method further comprises administering an immunotherapy, optionally anti-CTLA4 and/or anti-PD-1 checkpoint blockade immunotherapy. In some embodiments, the chemotherapeutic agent moiety is covalently conjugated to the nanoparticle or the covalent conjugation of the nanoparticle to the chemotherapeutic agent moiety improves drug stability, prolongs circulation half-life, enhances drug delivery, or enhances treatment outcomes. In some embodiments, the CCR2-targeting composition eliminates CCR2+ myeloid cells or inhibits tumor growth. In some embodiments, the CCR2-targeting composition is Cu- or ⁶⁴Cu-CuNPs-ECL1 i-Gem and is an imaging agent and a therapeutic agent for PDAC. In some embodiments, administration of the CCR2-targeting composition results in tumor necrosis, effective inhibition of tumor growth, or prolonged survival. In some embodiments, administration of the CCR2-targeting composition results in at least about 20% extended survival. In some embodiments, the CCR2-targeting composition inhibits tumor-associated macrophages recruitment by targeting CCR2. In some embodiments, the CCR2-targeting composition is a CCR2-targeting ultrasmall nanoparticle for PDAC imaging and/or therapy. In some embodiments, an immunotherapy (e.g., a checkpoint inhibitor) is administered before, after, simultaneously, or consecutively with the CCR2-targeting composition. In some embodiments, the CCR2-targeting composition is a copper nanocluster (CuNC), wherein the CuNC is biodegradable, or the degradation of CuNC enables the release of the chemotherapeutic agent (e.g., gemcitabine (Gem)) within a tumor microenvironment (TME) to kill the CCR2+ stromal cells including tumor-infiltrating inflammatory monocytes (IMs) or macrophages (TAMs) to change the TME to immunoresponsive. In some embodiments, the CCR2-targeting composition inhibits growth of a tumor or prolongs survival. In some embodiments, the cancer is pancreatic cancer (e.g., PDAC). In some embodiments, the method further comprises PET imaging for PET-guided delivery of the CCR2-targeting composition.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A-FIG. 1D. Synthesis and characterization of ultrasmall copper nanoparticles (CuNPs). (A) Schematic diagram of the synthesis of CuNPs-ECL1i, CuNPs-ECL1i-Gem, and 64Cu radiolabeled nanoparticles, (B) STEM of CuNPs-ECL1 i, (C) Number average hydrodynamic diameter of CuNPs-ECL1i, (D) In vitro TA-PEG-Gem release profiles of CuNPs-ECL1 i-Gem under physiological and acidic conditions.

FIG. 2A-FIG. 2C. Biodistribution of 64Cu-CuNPs-ECL1i and 64Cu-CuNPs-ECL1 i-Gem in wild type C57BL/6 mice (n=4/group) at (A) 1 h, (B) 4 h and (C) 24 h post injection.

FIG. 3A-FIG. 3E. (A) Representative In vivo PET/CT images of 64Cu-CuNPs-ECL1 i in wild type littermate, 7-9-week-old KPPC mice, KPPC mice with 50-fold blocking dose, and 64Cu-CuNPs-NT (non-targeted 64CuNPs) in KPPC mice at 24 h post injection (yellow arrow: pancreas/pancreatic tumor). Quantitative analysis of (B) tumor uptakes and (C) tumor/muscle (T/M) ratio that showed significant difference between KPPC mice, wild type littermates, blocking mice, and 64Cu-CuNPs-NT (**** p<0.0001, n=4-5/group). (D) Autoradiography of 64Cu-CuNPs-ECL1 i uptake in KPPC tumor showing intense and heterogeneous distribution compared to the low accumulation in a wild type mouse. (E) H&E, trichrome, CCR2 (brown), and control IgG immunostaining showing the histology, fibrosis, and CCR2 expression of the KPPC tumor.

FIG. 4A-FIG. 4D. (A) Representative In vivo PET/CT images of 64Cu-CuNPs-ECL1i in 22-23-week-old KPC mice and wild type littermates, and 64Cu-CuNPs-NT in KPC mice at 24 h post injection (yellow arrow: pancreas/pancreatic tumor). Quantitative analysis of (B) tumor uptakes and (C) tumor/muscle ratio of 64Cu-CuNPs-ECL1i in WT and KPC mice, and 64Cu-CuNPs-NT in KPC mice showing significant difference among KPC mice, wild type littermate, and 64Cu-CuNPs-NT (*** p<0.001, **** p<0.0001, n=4-5/group). (D) Autoradiography showing intensive but heterogeneous 64Cu-CuNPs-ECL1 i distribution across the KPC tumor. H&E, and trichrome staining of KPC tumor showing the correlation between fibrotic regions (blue) and 64Cu-CuNPs-ECL1 i tumor uptake.

FIG. 5A-FIG. 5E. The tumor growth (A) and mouse survival (B) curves of KI implanted mice after treating with CuNPs-ECL1 i-Gem, CuNPs-ECL1i, CuNPs-Gem, gemcitabine (7 mg/kg body weight, I.V.), gemcitabine (100 mg/kg body weight, I.P.), and saline. 1st treatment with CuNPs-ECL1 i-Gem started at 10 days post tumor implantation. The CuNPs-Gem and 2nd treatment began at 7 days post tumor implantation. H&E staining of the tumor slices from mice treated with CuNPs-ECL1 i-Gem (C), gemcitabine (100 mg/kg body weight) (D), and saline (E).

FIG. 6A-FIG. 6C. Hematological (A) and serum biochemical (B) tests of C57BL/6 mice after 2 weeks receiving successive treatment (twice per week) of 10 mg/kg (Cu dose) CuNPs-ECL1i and CuNPs-ECL1 i-Gem, and 100 μL saline for control group. (C) The H&E staining of bone marrow, liver, and kidney showed no significant histological differences between CuNPs-ECL1 i-Gem and the control group.

FIG. 7 is a schematic depicting injection of a mouse with CCR2-targeted gemcitabine nanoparticles and the resulting effect on survival.

FIG. 8 is a schematic depicting synthesis of TA-PEG-ECL1 i and TA-PEG-Gem.

FIG. 9 is a MALDI-TOF mass spectrum of TA-PEG-ECL1 i.

FIG. 10 is a 1^{H} NMR and MALDI-TOF mass spectrum of TA-PEG-Gem.

FIG. 11 is a HPLC spectra of TA-PEG-ECL1i (red, monitored at 210 nm), TA-PEG-Gem (green, monitored at 254 nm), and CuNPs-ECLi1-Gem after dissolution by 0.1 M HCl (blue, monitored at 254 nm; black, monitored at 210 nm).

FIG. 12 is a FPLC analysis of CuNPs-ECL1i.

FIG. 13 is a radio-TLC of ⁶⁴Cu-CuNPs-ECL1i.

FIG. 14 is a graph depicting cell viability of Gem and TA-PEG-Gem in KI cells showing comparable IC₅₀ of 19.7 μM and 48.8 μM, respectively.

FIG. 15A-FIG. 15C. In vitro cell uptake of ⁶⁴Cu-CuNPs-NT and ⁶⁴Cu-CuNPs-ECL1i in (A) THP1 cells and (B) KI cells. (C) Immunofluorescence staining showing CCR2 (green) expression in the KI cells.

FIG. 16A-FIG. 16B. (A) PET/CT imaging and (B) the quantitative analysis of ⁶⁴Cu-CuNPs-ECL1 i blocked with 50 times of CuNPs-NT in 7-week-old KPPC mice (n=4) in which the tumor uptake was comparable with that of ⁶⁴Cu-CuNPs-ECL1 i in FIG. 3 (red column).

FIG. 17 is a series of images depicting amplified H&E and trichrome staining of the radioactive regions in autoradiography of KPC tumor slide showing correlation between fibrotic regions and ₆₄Cu-CuNPs-ECL1i tumor uptake.

FIG. 18A-FIG. 18E. ¹⁸F-FDG PET/CT characteristics in PDAC models. (A) In vivo PET/CT images of ¹⁸F-FDG at 1 h post injection in KPPC, KPC mice, and wild type littermates, respectively (yellow arrow: pancreas/pancreatic tumor). Quantitative analysis of (B) tumor uptakes and (C) tumor/muscle ratio of 18^{F}-FDG in KPPC, KPC mice, and wild type littermates. Comparison of tumor uptake (D) and tumor/muscle ratio (E) of ⁶⁴Cu-CuNPs-ECL1i and ¹⁶F-FDG (* p<0.05, **** p<0.0001, n=3-4/group).

FIG. 19A-FIG. 19C. In vivo toxicity evaluation of CuNPs in (A) different doses (CD1 mice, n=5/group), and the comparison of the (B) weight and (C) percentage of body weight of liver and kidneys.

FIG. 20 is a series of images depicting that H&E staining of heart and lung showed no significant histological differences between CuNPs-ECL1 i-Gem and the control group.

FIG. 21A-FIG. 21C. Characterization of CCR2 targeting directed nanoimmunotherapy in KI PDAC tumor mice. (A) Tumor growth curves. (B) Bioluminescent imaging of mice treated with CuNC-ECL1 i-Gem and antibodies vs. CuNC-ECL1 i-Gem. The bioluminescent signal scale bar for CuNC-ECL1i-Gem treated mice was 2.3×10⁴ times higher than that of CuNC-ECL1 i-Gem and antibodies treated mice. (C) Mice survival curve.

FIG. 22A-FIG. 22B. CCR2 targeted nanoimmunotherapy of rechallenged tumors. (A) tumor size curve showing the development of 1^{st} and 2^{nd} implanted tumors and the variation following treatment. (B) Survival curve showing 100% survival during the study.

FIG. 23A-FIG. 23B. Assessment of roles of each immune checkpoint inhibitor in nanoimmunotherapy. (A) Tumor size curves. (B) KI tumor mice survival curves.

FIG. 24. Favorable intratumoral immune cells changes following treatment with CuNPs-ECL1 i-GEM. (A) Tumor weight graphs. (B) CD11 b⁺ cells, TAM, and Eosinphils graphs. (C) CD3⁺ T cells, CD8⁺ T cells, and CD8⁺CD44^{high} CD6^{low} effector T cells graphs.

### DETAILED DESCRIPTION OF THE INVENTION

The present disclosure is based, at least in part, on the discovery that CuNC-ECL1 i-Gem is useful for pancreatic cancer treatment and ⁶⁴CuNC-ECL1i-Gem is useful for treatment and PET imaging.

An immunotherapeutic agent can be added to the nanocluster agent to synergistically enhance efficacy and anti-tumor activity.

As shown herein, the present disclosure reports a C-C chemokine type 2 receptor (CCR2) targeting mediated nanoimmunotherapy to treat pancreatic ductal adenocarcinoma (PDAC). Also disclosed herein is the use of a biodegradable copper nanocluster (CuNC) for enhanced loading of chemotherapeutic drug Gemcitabine (Gem). Through the conjugation of a CCR2 binding peptide called ECL1 i on the surface of CuNC, the targeted CuNC-ECL1i-Gem can be specifically delivered to inflammatory monocytes (IMs) and tumor-associated macrophages (TAMs) within PDAC tumor microenvironment (TME), which are the major components to generate immunosuppressive TME to damp the treatment efficiency of immunotherapy. The degradation of CuNC enables the release of Gem within TME to kill the CCR2+ stromal cells including IMs and TAMs to change the TME to immunoresponsive. Following anti-CTLA4 and anti-PD-1 checkpoint blockade immunotherapy, the KI tumor-bearing mice demonstrated a complete response to the combined treatment in contrast to other treatment regimens including Gem alone in low and high doses, CuNCs-ECL1i, CuNCs-ECL1 i-Gem, and anti-CTLA4+anti-PD-1 treatment.

Taken together, in contrast to conventional chemotherapy and immunotherapy, which showed no therapeutic efficiency in this KI mouse PDAC model, the disclosed strategy demonstrated removal of therapeutic efficiency in this KI mouse PDA tumors with 100% survival during the study.

Cancer

Methods and compositions as described herein can be used for the imaging, prevention, treatment, or slowing the progression of cancer or tumor growth (e.g., especially in advanced cancer, metastasis, and relapse or CCR2/CCL2-associated cancers (or cancers overexpressing CCR2, cancer cells expressing CCR2), such as breast, squamous cell, ovarian, cervical, sarcoma, esophageal, gastric, renal cell, lung, colon, colorectal, pepillary thyroid, and prostate cancer). For example, the cancer can be Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bladder Cancer; Bone Cancer (including Ewing Sarcoma and Osteosarcoma and Malignant Fibrous Histiocytoma); Brain Tumors; Breast Cancer; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Intraocular Melanoma; Intraocular Melanoma; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer; Intraocular Melanoma; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Lymphoma; Male Breast Cancer; Malignant Fibrous Histiocytoma of Bone or Osteosarcoma; Melanoma; Melanoma, Intraocular (Eye); Merkel Cell Carcinoma (Skin Cancer); Mesothelioma, Malignant; Metastatic Cancer; Metastatic Squamous Neck Cancer with Occult Primary; Midline Tract Carcinoma Involving NUT Gene; Mouth Cancer; Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip or Oral Cavity Cancer; Oropharyngeal Cancer; Osteosarcoma and Malignant Fibrous Histiocytoma of Bone; Ovarian Cancer Pancreatic Cancer; Pancreatic Neuroendocrine Tumors (Islet Cell Tumors); Papillomatosis; Paraganglioma; Paranasal Sinus and Nasal Cavity Cancer; Parathyroid Cancer; Penile Cancer; Pharyngeal Cancer; Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer; Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sezary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; T-Cell Lymphoma, Cutaneous; Lymphoma; Mycosis Fungoides and Sézary Syndrome; Testicular Cancer; Throat Cancer; Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer; Urethral Cancer; Uterine Cancer, Endometrial; Uterine Sarcoma; Vaginal Cancer; Vascular Tumors (Soft Tissue Sarcoma); Vulvar Cancer; or Wilms Tumor.

CCR2-Targeting Composition: Anti-Cancer/Imaging Agents

The CCR2-targeting composition can be an anti-cancer composition and/or imaging agent. The CCR2-targeting composition can comprise a CCR2 binding peptide comprising a TFLK sequence; an anti-cancer or chemotherapeutic moiety (e.g., a chemotherapeutic agent, an immune checkpoint inhibitor, a radiopharmaceutical, etc.), and/or a nanoparticle.

CCR2 Binding Peptide

As described herein, the imaging agent comprises a CCR2 binding peptide. The CCR2 binding peptide can be any peptide with CCR2 activity. For example, the CCR2 binding peptide can be a CCR2 non-competitive antagonist peptide. As another example, the CCR2 binding peptide can be a cyclized CCR2 binding peptide.

As described herein, monocyte chemo-attractant protein chemokines are secreted by a wide variety of cell types under a range of inflammatory conditions such as atherosclerosis, neurodegenerative disease, and various forms of cancer (among various other diseases and conditions). One of the most prominent chemokines is monocyte chemoattractant protein-1 (MCP-1), now called CCL2, which significantly regulates migration and infiltration of monocytes to the site of inflammation, predominantly through CC-chemokine receptor 2 (CCR2).

CCR2 binding peptides can be any CCR2 peptide known, such as those described in US Pat Pub No. 2015/0011477 and US Pat Pub No. 2017/0348442, incorporated herein by reference in their entireties. The imaging agent as described herein comprises a CCR2 binding peptide. For example, the CCR2 binding peptide can comprise an ECL1 i peptide, as described in US Pat Pub No. 2015/0011477 and US Pat Pub No. 2017/0348442, incorporated herein by reference. Because CCR2+ cells migrate in response to CCL2, CCR2 can be a surrogate marker for CCR2.

For example, the CCR2 binding peptide can comprise ECL1 i. An ECL1i peptide can be of the sequence DLeu-Gly-DThr-DPhe-DLeu-DLys-DCys (SEQ ID NO: 3). As another example, a CCR2 binding peptide can be a peptide comprising the following amino acid sequence Thr-Phe-Leu-Lys or Thr-Phe-Leu-Lys-Cys, useful as a CCR2 non-competitive antagonist peptide. As another example, the ECL1i peptide can be a cyclized ECL1i peptide (e.g., Cyclo-(Orn-LGTFLK)).

Among all the peptides tested, the heptapeptide LGTFLKC, named ECL1 (C) inverso, presented interesting properties as a CCR2 non-competitive antagonist peptide. This peptide corresponds to an inverted sequence in the third transmembrane domain of CCR2, more precisely in the juxtamembranous and N-terminal region of the third transmembrane domain. In some embodiments, all or part of the amino acids are in a D configuration.

As another example, as described in US Pat Pub No. 2015/0011477 (incorporated by reference, herein), the CCR2 binding peptide can be:

### CCR2 Binding Peptides:

| SEQ | | | |
|---|---|---|---|
| ID NO: | Description | Peptide Sequence | Comments |
| 1 | | Thr Phe Leu Lys Cys | |
| 2 | | Xaa Thr Phe Leu Lys | Xaa in position 1 is absent, |
| | | Cys Xaa | is glycine or represents an |
| | | | amino acid sequence selected |
| | | | from the group consisting of |
| | | | LG, YLG, and HYLG; “Xaa in |
| | | | position 7” independently is |
| | | | absent, is methionine, or |
| | | | represents an amino acid |
| | | | sequence selected from the |
| | | | group consisting of MA, MAN, |
| | | | MANG, MANGF, MANGFV, |
| | | | MANGFVW, MANGFVWE, |
| | | | and MANGFVWEN |
| 3 | ECL1 (C) | Leu Gly Thr Phe Leu | |
| | inverso; | Lys Cys | |
| | ECL1i | | |
| 4 | | His Tyr Leu Gly Thr | |
| | | Phe Leu Lys Cys Met | |
| | | Ala | |
| 5 | | Leu Gly Thr Phe Leu | |
| | | Lys Cys Met Ala | |
| 6 | | His Tyr Leu Gly Thr | |
| | | Phe Leu Lys Cys | |
| 7 | | Gly Thr Phe Leu Lys | |
| | | Cys Met Ala Asn Gly | |
| | | Phe | |
| 8 | | Thr Phe Leu Lys Cys | |
| | | Met Ala Asn Gly Phe | |
| | | Val | |
| 9 | | His Tyr Leu Gly Thr | |
| | | Phe Leu Lys Cys Met | |
| | | Ala Asn Gly Phe Val | |
| | | Trp | |
| 10 | ECL1 (C) | Cys Lys Leu Phe Thr | |
| | | Gly Leu | |
| 11 | ECL2 (N) | Leu Phe Thr Lys Cys | |
| 12 | ECL2 (N) | Cys Lys Thr Phe Leu | |
| | inverso | | |
| 13 | ECL3 (C) | His Thr Leu Met Arg | |
| | | Asn Leu | |
| 14 | ECL3 (C) | Leu Asn Arg Met Leu | |
| | inverso | Thr His | |
| 15 | ECL3 (N) | Leu Asn Thr Phe Gln | |
| | | Glu Phe | |
| 16 | ECL3 (N) | Phe Glu GIn Phe Thr | |
| | inverso | Asn Leu | |
| 17 | | Thr Phe Leu Lys | |
| 18 | | Xaa Thr Phe Leu Lys | Xaa in position 1 is absent, |
| | | Xaa | is glycine or represents an |
| | | | amino acid sequence selected |
| | | | from the group consisting of |
| | | | AG, LG, YLG, and HYLG; |
| | | | “Xaa in position 6” |
| | | | independently is absent or |
| | | | is alanine |
| 19 | | Leu Gly Thr Phe Leu | |
| | | Lys | |
| 20 | | Ala Gly Thr Phe Leu | |
| | | Lys Cys | |
| 21 | | Leu Gly Thr Phe Leu | |
| | | Lys Ala | |
| 22 | | Gly Thr Phe Leu Lys | |
| 23 | | Ala Gly Thr Phe Leu | |
| | | Lys Ala | |
| 24 | | Met Ala Asn Gly | |
| 25 | | Met Ala Asn Gly Phe | |
| 26 | | Met Ala Asn Gly Phe | |
| | | Val | |
| 27 | | Met Ala Asn Gly Phe | |
| | | Val Trp | |
| 28 | | Met Ala Asn Gly Phe | |
| | | Val Trp Glu | |
| 29 | | Met Ala Asn Gly Phe | |
| | | Val Trp Glu Asn | |
| 30 | | His Tyr Leu Gly | |

The CCR2 binding peptide as described herein can comprise an amino acid length of about 4 amino acids to about 200 amino acids or about 4 amino acids to about 50 amino acids. For example, the CCR2 biding peptide can comprise an amino acid length of no more than 4 amino acids; 5 amino acids; 6 amino acids; 7 amino acids; 8 amino acids; 9 amino acids; 10 amino acids; 11 amino acids; 12 amino acids; 13 amino acids; 14 amino acids; 15 amino acids; 16 amino acids; 17 amino acids; 18 amino acids; 19 amino acids; 20 amino acids; 21 amino acids; 22 amino acids; 23 amino acids; 24 amino acids; 25 amino acids; 26 amino acids; 27 amino acids; 28 amino acids; 29 amino acids; 30 amino acids; 31 amino acids; 32 amino acids; 33 amino acids; 34 amino acids; 35 amino acids; 36 amino acids; 37 amino acids; 38 amino acids; 39 amino acids; 40 amino acids; 41 amino acids; 42 amino acids; 43 amino acids; 44 amino acids; 45 amino acids; 46 amino acids; 47 amino acids; 48 amino acids; 49 amino acids; 50 amino acids; 51 amino acids; 52 amino acids; 53 amino acids; 54 amino acids; 55 amino acids; 56 amino acids; 57 amino acids; 58 amino acids; 59 amino acids; 60 amino acids; 61 amino acids; 62 amino acids; 63 amino acids; 64 amino acids; 65 amino acids; 66 amino acids; 67 amino acids; 68 amino acids; 69 amino acids; 70 amino acids; 71 amino acids; 72 amino acids; 73 amino acids; 74 amino acids; 75 amino acids; 76 amino acids; 77 amino acids; 78 amino acids; 79 amino acids; 80 amino acids; 81 amino acids; 82 amino acids; 83 amino acids; 84 amino acids; 85 amino acids; 86 amino acids; 87 amino acids; 88 amino acids; 89 amino acids; 90 amino acids; 91 amino acids; 92 amino acids; 93 amino acids; 94 amino acids; 95 amino acids; 96 amino acids; 97 amino acids; 98 amino acids; 99 amino acids; 100 amino acids; 101 amino acids; 102 amino acids; 103 amino acids; 104 amino acids; 105 amino acids; 106 amino acids; 107 amino acids; 108 amino acids; 109 amino acids; 110 amino acids; 111 amino acids; 112 amino acids; 113 amino acids; 114 amino acids; 115 amino acids; 116 amino acids; 117 amino acids; 118 amino acids; 119 amino acids; 120 amino acids; 121 amino acids; 122 amino acids; 123 amino acids; 124 amino acids; 125 amino acids; 126 amino acids; 127 amino acids; 128 amino acids; 129 amino acids; 130 amino acids; 131 amino acids; 132 amino acids; 133 amino acids; 134 amino acids; 135 amino acids; 136 amino acids; 137 amino acids; 138 amino acids; 139 amino acids; 140 amino acids; 141 amino acids; 142 amino acids; 143 amino acids; 144 amino acids; 145 amino acids; 146 amino acids; 147 amino acids; 148 amino acids; 149 amino acids; 150 amino acids; 151 amino acids; 152 amino acids; 153 amino acids; 154 amino acids; 155 amino acids; 156 amino acids; 157 amino acids; 158 amino acids; 159 amino acids; 160 amino acids; 161 amino acids; 162 amino acids; 163 amino acids; 164 amino acids; 165 amino acids; 166 amino acids; 167 amino acids; 168 amino acids; 169 amino acids; 170 amino acids; 171 amino acids; 172 amino acids; 173 amino acids; 174 amino acids; 175 amino acids; 176 amino acids; 177 amino acids; 178 amino acids; 179 amino acids; 180 amino acids; 181 amino acids; 182 amino acids; 183 amino acids; 184 amino acids; 185 amino acids; 186 amino acids; 187 amino acids; 188 amino acids; 189 amino acids; 190 amino acids; 191 amino acids; 192 amino acids; 193 amino acids; 194 amino acids; 195 amino acids; 196 amino acids; 197 amino acids; 198 amino acids; 199 amino acids; or 200 amino acids. Recitation of each of these discrete values is understood to include ranges between each value. Recitation of each of a range is understood to include discrete values within the range.

Radiolabel

One embodiment of the present disclosure provides for a radiolabeled composition or a composition with a radionuclide. In some embodiments, the nanoparticle can comprise a radiolabel, but it is also envisioned that the nanoparticle can be loaded with a radiolabel or a radiolabel can be conjugated to the surface of the nanoparticle. For example, the radiolabel can provide radiotherapy to a tumor or cancer or the radiolabel can be detected for use as an imaging agent.

The CCR2-targeting composition (e.g., imaging agent or anti-cancer composition), as described herein, can comprise a radiolabel (also known as a radionuclide). Radiolabeling processes are also described in Example 1; see e.g. Fani et al. Theranostics 2012; 2(5):481-501. doi:10.7150/thno.4024. Except as otherwise noted herein, therefore, the process of the present disclosure can be carried out in accordance with such processes.

One embodiment of the present disclosure provides for a radiolabeled peptide. According to another embodiment, the radiolabeled compound can be an imaging agent or a radiotherapeutic.

References herein to “radiolabeled” include a compound where one or more atoms are replaced or substituted by an atom having an atomic mass or mass number different from the atomic mass or mass number typically found in nature (i.e., naturally occurring). One non-limiting exception is ¹⁹F, which allows detection of a molecule that contains this element without enrichment to a higher degree than what is naturally occurring. Compounds carrying the substituent ¹⁹F may thus also be referred to as “labeled” or the like. The term radiolabeled may be interchangeably used with “isotopically-labeled”, “labeled”, “isotopic tracer group”, “isotopic marker”, “isotopic label”, “detectable isotope”, or “radioligand”.

In one embodiment, the compound comprises a single radiolabeled group. Examples of suitable, non-limiting radiolabel groups can include: ²H (D or deuterium), ³H (T or tritium), ¹¹C, ¹³C, ¹⁴C, ⁶⁴Cu, ⁶⁷Cu, ¹⁷⁷Lu, ¹³N, ¹⁵N, ¹⁵O, ¹⁷O, ¹⁸O, ¹⁸F, ⁸⁹Sr, ³⁵S ¹⁵³Sm, ³⁶Cl, ⁸²Br, ⁷⁵Br, ⁷⁶Br, ⁷⁷Br, ¹²³I, ¹²⁴I, ¹²⁵I, ¹³¹I, ¹¹¹In, ⁶⁷Ga, ⁶⁸Ga, ¹⁷⁷Lu, ¹⁸⁶Re, ¹⁸⁸Re, ²⁰¹Tl ^{99m}Tc, ⁹⁰Y, or ⁸⁹Zr. It is to be understood that an isotopically labeled compound needs only to be enriched with a detectable isotope to, or above, the degree which allows detection with a technique suitable for the particular application, e.g., in a detectable compound labeled with ¹C, the carbon-atom of the labeled group of the labeled compound may be constituted by ¹²C or other carbon-isotopes in a fraction of the molecules. The radionuclide that is incorporated in the radiolabeled compounds will depend on the specific application of that radiolabeled compound. For example, “heavy” isotope-labeled compounds (e.g., compounds containing deuterons/heavy hydrogen, heavy nitrogen, heavy oxygen, heavy carbon) can be useful for mass spectrometric and NMR-based studies. As another example, for in vitro labeling or in competition assays, compounds that incorporate ³H, ¹⁴C, or ¹²⁵I can be useful. For in vivo imaging applications ¹¹C, ¹³C, ¹⁸F, ¹⁹F, ¹²⁰I, ¹²³I, ¹³¹I, ⁷⁵Br, or ⁷⁶Br can generally be useful. In one embodiment, the radiolabel is ⁶⁴Cu.

In some embodiments, the radioisotope comprises a positron-emitting isotope suitable for use in PET imaging. For example, the radioisotope can comprise a synthetic radioisotope. For example, the radioisotope can be a positron emitter selected from ¹⁴O, ¹⁵O, ¹³N, ¹¹C, ¹⁸F, ²²Na, ²⁶Al, ⁸²Rb, ³⁸K, ⁶²Cu, ⁶³Zn, ⁷⁰As, ⁶⁸Ga, ⁶¹Cu, ⁵²Fe, ⁶²Zn, ⁶³Zn, ⁶⁴Cu, ⁸⁶Y, ⁷⁶Br, ⁵⁵Co, ⁷¹As, ⁷⁴As, ⁶⁸Ge, ⁴⁰K, ¹²¹I, ¹²⁰I, ¹¹⁰In, ⁹⁴Tc, ¹²²Xe, ⁸⁹Zr, or ¹²⁴I. In some embodiments, the radioisotope is ⁶⁴Cu.

As another example, the imaging agent comprising a radiolabel can comprise Oxygen-15 water, Nitrogen-13 ammonia, [⁸²Rb] Rubidium-82 chloride, [¹¹C], [¹¹C] 25B-NBOMe, [¹⁸F] Altanserin, [¹¹C] Carfentanil, [¹¹C] DASB, [¹¹C]DTBZ, [¹⁸F]Fluoropropyl-DTBZ, [¹¹C] ME@HAPTHI, [¹⁸F] Fallypride, [¹⁸F]Florbetaben, [¹⁸F] Flubatine, [¹⁸F] Fluspidine, [¹⁸F] Florbetapir, [¹⁸F] or [¹¹C]Flumazenil, [¹⁸F] Flutemetamol, [¹⁸F] Fluorodopa, [¹⁸F] Desmethoxyfallypride, [¹⁸F] Mefway, [¹⁸F] MPPF, [¹⁸F] Nifene, Pittsburgh compound B, [¹¹C] Raclopride, [¹⁸F] Setoperone, [¹⁸F] or [¹¹C] N-Methylspiperone, [¹¹C] Verapamil, [¹¹C]Martinostat, Fludeoxyglucose (¹⁸F)(FDG)-glucose analogue, [¹¹C] Acetate, [¹¹C]Methionine, [¹¹C] Choline, [¹⁸F] Fluciclovine, [¹⁸F] Fluorocholine, [¹⁸F] FET, [¹⁸F]FMISO, [¹⁸F] 3′-fluoro-3′-deoxythymidine, [⁶⁸Ga] DOTA-pseudopeptides, [⁶⁸Ga]PSMA, or [¹⁸F] Fluorodeoxysorbitol (FDS).

Nanoparticle

As described herein, a radiolabel can be doped in or on a nanoparticle, or a radiolabel can be conjugated to a nanoparticle. In an aspect of the present disclosure the nanoparticle is a copper (optionally comprising ⁶⁴Cu) nanoparticle.

The composition, as described herein can comprise any nanoparticle known in the art suitable for use as a CCR2-targeting anti-cancer/imaging agent.

Nanoparticles for use in molecular probes and imaging agents are well known; see e.g., Chen et al., Molecular Imaging Probes for Cancer Research, 2012.

Labeling of nanoparticles are well known; see e.g., Yongjian Liu, Michael J Welch, Nanoparticles labeled with positron emission nuclides: advantages, methods, and applications, Bioconjugate Chemistry, 2012, 23, 671-682; Stockholf et al., Pharmaceuticals (Basel). 2014 April; 7(4): 392-418. Except as otherwise noted herein, therefore, the process of the present disclosure can be carried out in accordance with such processes.

For example, a nanoparticle can be a nanocluster or any other type of nanostructures including organic, inorganic, or lipid nanostructures.

As another example, the nanoparticle can comprise Au or Cu. As another example, the nanoparticle can comprise iron oxide, gold, gold nanoclusters (AuNC), gold nanorods (AuNR), copper (Cu), quantum dots, carbon nanotubes, carbon nanohorn, gadolinium (Gd), dendrimers, dendrons, polyelectrolyte complex (PEC) nanoparticles, calcium phosphate nanoparticles, perfluorocarbon nanoparticles (PFCNPs), or lipid-based nanoparticles, such as liposomes and micelles.

Linker

Described herein are linkers used to attach peptides to a portion of an imaging agent (e.g., a core, a nanoparticle, a radiolabel, a chelator, another peptide). A linker can be any composition used for conjugation, for example to a nanoparticle or chelator. In an aspect of the present disclosure, the linker is PEG (or a PEG derivative).

A linker group can be any linker group suitable for use in an imaging agent. Linker groups for imaging agents (e.g., molecular probes) are well known (see e.g., Werengowska-Ciedwierz et al., Advances in Condensed Matter Physics, Vol. 2015 (2015); Chen et al., Curr Top Med Chem. 2010; 10(12): 1227-1236). Except as otherwise noted herein, therefore, the processes of the present disclosure can be carried out in accordance with such processes.

For example, the linker can conjugate a nanoparticle to a CCR2 binding peptide. For example, the CCR2 binding peptide can be covalently attached to the linker. For example, the linker can comprise a poly(ethylene glycol) (PEG) derivative. As another example, the linker can comprise PEG, TA-PEG-Maleimide, TA-PEG-OMe, or TA-PEG. As another example, a linker can comprise an isothiocyanate group, a carboxylic acid or carboxylate groups, a dendrimer, a dendron, Fmoc-protected-2,3-diaminopropanoic acid, ascorbic acid, a silane linker, minopropyltrimethoxysilane (APTMS), or dopamine. Other covalent coupling methods can use employ the use of 2 thiol groups, 2 primary amines, a carboxylic acid and primary amine, maleimide and thiol, hydrazide and aldehyde, or a primary amine and aldehyde. For example, the linker can be an amide, a thioether, a disulfide, an acetyl-hydrazone group, a polycyclic group, a click chemistry (CC) group (e.g., cycloadditions, for example, Huisgen catalytic cycloaddition; nucleophilic substitution chemistry, for example, ring opening of heterocyclic electrophiles; carbonyl chemistry of the “nonaldol” type, for example, formation of ureas, thioureas, and hydrazones; additions to carbon-carbon multiple bonds, for example, epoxidation and dihydroxylation); or a physical or chemical bond.

Anti-Cancer Agent Moiety

Chemotherapeutic Agent

In some embodiments, the anti-cancer moiety conjugated to the nanoparticle of the CCR2-targeting composition can comprise a chemotherapeutic agent.

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

In some embodiments, the agents described herein can be used in combination with chemotherapeutic agents or used to sensitize a tumor, subject, or cancer to a chemotherapeutic agent.

Examples of chemotherapeutic agents include alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1 and calicheamicin ω1; dynemicin, including dynemicin A; uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-1-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, or zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichloro-triethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; mitoxantrone; teniposide; edatrexate; daunomycin; aminopterin; capecitabine; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosourea, dactinomycin, daunorubicin, doxorubicin, bleomycin, plicomycin, mitomycin, etoposide (VP16), tamoxifen, raloxifene, estrogen receptor binding agents, paclitaxel, docetaxel, gemcitabine, vinorelbine, farnesyl-protein transferase inhibitors, transplatinum, 5-fluorouracil, vincristine, vinblastine, or methotrexate or pharmaceutically acceptable salts, acids or derivatives of any of the above.

Other examples of chemotherapeutic agents can be Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alkeran (Melphalan Hydrochloride), Alkeran (Melphalan), Alimta (Pemetrexed Disodium), Aloxi (Palonosetron Hydrochloride), Ambochlorin/Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar (Irinotecan Hydrochloride), Capecitabine, CAPOX, Carac (Fluorouracil-Topical), Carboplatin, Carboplatin-Taxol, Carfilzomib, Carmubris (Carmustine), Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, Chlorambucil-prednisone, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil-Topical, Flutamide, Folex (Methotrexate), Folex PFS (Methotrexate), FOLFIRI, FOLFIRI-bevacizumab, FOLFIRI-Cetuximab, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, Gemcitabine-Cisplatin, Gemcitabine-Oxaliplatin, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), IL-2 (Aldesleukin), Imatinib Mesylate, Imbruvica (Ibrutinib), Imiquimod, Imlygic (Talimogene Laherparepvec), Inlyta (Axitinib), Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituximab, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tolak (Fluorouracil-Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VelP, Velban (Vinblastine Sulfate), Velcade (Bortezomib), Velsar (Vinblastine Sulfate), Vemurafenib, Venclexta (Venetoclax), Venetoclax, Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zelboraf (Vemurafenib), Zevalin (lbritumomab Tiuxetan), Zinecard (Dexrazoxane Hydrochloride), Ziv-Aflibercept, Zoladex (Goserelin Acetate), Zoledronic Acid, Zolinza (Vorinostat), Zometa (Zoledronic Acid), Zydelig (Idelalisib), Zykadia (Ceritinib), or Zytiga (Abiraterone Acetate) or pharmaceutically acceptable salts, acids or derivatives of any of the above.

Immunotherapy

As described herein, the compositions and agents described herein can be used in combination with a number of therapies, such as immunotherapy (e.g., CAR-T) or checkpoint immunotherapy. It is envisioned that the compositions described herein can be used in combination with immunotherapy, but can also be conjugated with an immunotherapeutic agent (e.g., checkpoint inhibitors).

Immunotherapies are a new generation of cancer therapy that has revolutionized the treatment of otherwise terminal cancers, often achieving durable, sustained remission in cancers that were otherwise thought to be refractory to standard first- and second-line therapies. Thousands of patients annually are now treated with these life-saving therapies.

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and natural killer (NK) cells. The combination of therapeutic modalities, i.e., direct cytotoxic activity and inhibition or reduction of ErbB2 would provide therapeutic benefit in the treatment of ErbB2 overexpressing cancers.

Examples of immunotherapy can be immune effector cell (IEC) therapy (e.g., CAR T, mesenchymal stem cells) or T cell engaging therapy (e.g., CD19-specific T cell engager, such as blinatumomab, T cell engaging monoclonal antibody, bispecific T cell engager (BiTE) therapy).

In one aspect of immunotherapy, the tumor cell must bear some marker that is amenable to targeting, i.e., is not present on the majority of other cells. As described herein CCR2 is targeted. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides, et al., 1998), cytokine therapy, e.g., interferons α, β, and γ; IL-1, GM-CSF, TNF (Bukowski, et al., 1998; Davidson, et al., 1998; Hellstrand, et al., 1998) gene therapy, e.g., TNF, IL-1, IL-2, p53 (Qin et al., 1998; Austin-Ward and Villaseca, 1998; U.S. Pat. Nos. 5,830,880 and 5,846,945), and monoclonal antibodies, e.g., anti-ganglioside GM2, anti-HER-2, anti-p185 (Pietras, et al., 1998; Hanibuchi, et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton, et al., 1992; Mitchell, et al., 1990; Mitchell, et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg, et al., 1988; 1989).

CAR T

In some embodiments, the immunotherapy in accordance with the present disclosure is CAR T cell therapy (e.g., CD19-specific chimeric antigen receptor T (CAR-T)). Generally, CAR T cell therapy refers to any type of immunotherapy in which a subject's T cells are genetically modified to express chimeric antigen receptors. These chimeric antigen receptors allow the T cells to more effectively recognize and subsequently destroy cancer cells. Typically, T cells are first harvested from a subject, genetically altered to express a CAR targeting an antigen of interest (e.g., an antigen expressed on the surface of a tumor or cancer cell), and then infused back into the subject. Once infused into the subject, CAR T cells bind to the target antigen and are activated, allowing them to proliferate and become cytotoxic.

For example, the CAR T cell therapy can be any one or more of the currently FDA-approved CAR T cell therapies, which include anti-CD19 and anti-BCMA CAR T cells such as tisagenlecleucel (Kymriah), axicabtagene ciloleucel (Yescarta), brexucabtagene autoleucel (Tecartus), lisocabtagene maraleucel (Breyanzi), or idecabtagene vicleucel (Abecma). In some embodiments, the CAR T cell therapy is tisagenlecleucel or axicabtagene ciloleucel.

The CAR T cell therapy can also be a non-FDA-approved or experimental CAR T cell therapy (e.g., a CAR T cell therapy undergoing clinical trials), such as anti-CD22, anti-CAIX, anti-PSMA, anti-MUC1, anti-FRa, anti-meso-RNA, anti-CEA, anti-IL13Ra2, anti-HER2, or universal allogenic CAR T cells.

Checkpoint Immunotherapy

An important function of the immune system is its ability to tell between normal cells in the body and those it sees as “foreign.” This lets the immune system attack the foreign cells while leaving the normal cells alone. To do this, it uses “checkpoints.” Immune checkpoints are molecules on certain immune cells that need to be activated (or inactivated) to start an immune response.

Cancer cells can find ways to use these checkpoints to avoid being attacked by the immune system. But drugs that target these checkpoints hold a lot of promise as a cancer treatment. These drugs are called checkpoint inhibitors. Checkpoint inhibitors used to treat cancer don't work directly on the tumor at all. They only take the brakes off an immune response that has begun but hasn't yet been working at its full force.

Checkpoint immunotherapy has been extensively shown to unleash T cell effector functions to control tumors in many cancer patients. However, tumor cells can evade immunological elimination by recruiting myeloid cells that induce an immunosuppressive state. Recent high dimensional profiling studies have shown that tumor-infiltrating myeloid cells are considerably heterogeneous, and may include both immunostimulatory and immunosuppressive subsets, although they do not fit the M1/M2 paradigm. Thus, depletion of suppressive myeloid cells from tumors, blockade of their functions, or induction of myeloid cells with immunostimulatory properties may provide important approaches for improving immunotherapy strategies, perhaps in synergy with checkpoint blockade.

Any immune checkpoint inhibitor known in the art can be used. For example, a PD-1 inhibitor can be used. These drugs are typically administered IV (intravenously). PD-1 is a checkpoint protein on immune cells called T cells. It normally acts as a type of “off switch” that helps keep the T cells from attacking other cells in the body. It does this when it attaches to PD-L1, a protein on some normal (and cancer) cells. When PD-1 binds to PD-L1, it tells the T cell to leave the other cell alone. Some cancer cells have large amounts of PD-L1, which helps them hide from an immune attack.

Monoclonal antibodies that target either PD-1 or PD-L1 can block this binding and boost the immune response against cancer cells. These drugs have shown a great deal of promise in treating certain cancers.

Examples of drugs that target PD-1 can include: Pembrolizumab (Keytruda), Nivolumab (Opdivo), or Cemiplimab (Libtayo). These drugs have been shown to be helpful in treating several types of cancer, and new cancer types are being added as more studies show these drugs to be effective.

As another example, a PD-L1 inhibitor can be used. Examples of drugs that target PD-L1 can include: Atezolizumab (Tecentriq), Avelumab (Bavencio), or Durvalumab (Imfinzi). These drugs have also been shown to be helpful in treating different types of cancer, and are being studied for use against others.

CTLA-4 is another protein on some T cells that acts as a type of “off switch” to keep the immune system in check. For example, Ipilimumab (Yervoy) is a monoclonal antibody that attaches to CTLA-4 and reduces or blocks its function. This can boost the body's immune response against cancer cells. This drug can be used to treat melanoma of the skin and other cancers.

Bispecific Monoclonal Antibody (BsMAb)

In some embodiments, the immunotherapy in accordance with the present disclosure is bispecific monoclonal antibody (BsMAb) therapy. BsMAbs are synthetic proteins engineered to bind two different antigens simultaneously. For cancer immunotherapies, BsMAbs are typically designed to bind both a cytotoxic cell (e.g., a T cell) and an antigen expressed on a tumor or cancer cell. Engagement of T-cells and activation of antibody-dependent cellular cytotoxicity (ADCC) results in tumor cell death.

In some embodiments, the provided compositions and methods are used with, before, after, or in concurrence with any form of BsMAb therapy. For example, the BsMAb therapy can be any one or more of the currently FDA-approved BsMAb therapies, such as blinatumomab, emicizumab, or amivantamab

Cell Therapy

In cell therapy (also called cellular therapy, cell transplantation, or cytotherapy) viable cells can be injected, grafted, or implanted into a patient in order to effectuate a medicinal effect or therapeutic benefit. For example, transplanting T-cells capable of fighting cancer cells via cell-mediated immunity can be used in the course of immunotherapy.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogenic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter can repair themselves and function properly. It is envisioned that the compositions described herein can be used in combination with a radiotherapy, but can also be conjugated with a radiotherapeutic agent capable of emitting beta particles of sufficient energy, which are administered in a chemical form that reaches the tumor (e.g., ⁶⁴Cu, ¹³¹I). Some 3 particle-emitting radioisotopes such as ⁹⁰Y, ¹⁷⁷Lu, ¹⁸⁸Re, ¹³¹I, and ⁶⁴Cu have been studied for tumor-targeted radiotherapy. It has been widely recognized that high-energy β-particle-emitting radioisotopes such as ¹⁶⁸Re and ⁹⁰Y are suitable for treating larger tumors, while medium-low energy emission particles, such as ⁶⁴Cu, ¹⁵³Sm, and ¹⁷⁷Lu, are generally more suitable for treating small or metastatic tumors.

Radiation therapy used according to the present disclosure may include, but is not limited to, the use of γ-rays, X-rays, and/or the directed delivery of radioisotopes to tumor cells. As an example, the disclosed compositions can be radiolabeled such that the composition delivers the radiolabeled composition to a CCR2 expressing tumor cell. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 12.9 to 51.6 mC/kg for prolonged periods of time (3 to 4 wk), to single doses of 0.516 to 1.55 C/kg. Dosage ranges for radioisotopes vary widely and depend on the half-life of the isotope, the strength and type of radiation emitted, and the uptake by the neoplastic cells.

Radiotherapy may comprise the use of radiolabeled compositions or antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer or tumor. This ensures that a higher radiation dose is given to the tumor. Healthy surrounding cells and nearby structures receive a lower dose of radiation, so the possibility of side effects is reduced. A device called a multi-leaf collimator has been developed and may be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

Combination Therapies

The present disclosure may relate to one or more agents used in combination with an agent as described herein. The present disclosure describes combinations of a CCR2-targeting composition with other therapeutic modalities as combination therapies to increase the efficacy of anti-cancer therapies or as a stand alone cancer treatment.

To treat cancers using the methods and compositions of the present disclosure, one would generally administer to the subject a CCR2-targeting composition and optionally at least one other therapy. These therapies would be provided in a combined amount effective to achieve an increased activity, efficacy, cytotoxicity, or decrease off-target effects or dosage. This process may involve contacting the cells/subjects with both agents/therapies at the same time, e.g., using a single composition or pharmacological formulation that includes both agents, or by contacting the cell/subject with two distinct compositions or formulations, at the same time, wherein one composition includes the CCR2-targeting composition and the other includes the other agent.

Alternatively, the individual compounds in the compositions described herein may precede or follow the other compound treatment by time intervals ranging from seconds to days. One would generally ensure that a significant period of time did not expire between the time of each delivery, such that the therapies would still be able to exert an advantageously combined effect on the cell/subject. In such instances, it is contemplated that one would administer both modalities within about 12-24 hours of each other, within about 6-12 hours of each other, with a delay time of only about 1-2 hours, or less than 1 hour. Additionally, the CCR2-targeting composition may be administered about 10-15 minutes, about 5-10 minutes, or about 0-5 minutes prior to administration of the anti-cancer agent. For example, a CCR2-targeting composition may be administered from about 15 minutes, about 14 minutes, about 13 minutes, about 12 minutes, about 11 minutes, about 10 minutes, about 9 minutes, about 8 minutes, about 7 minutes, about 6 minutes, about 5 minutes, about 4 minutes, about 3 minutes, about 2 minutes, to about 1 minute, or any range derivable therein before or after an anti-cancer agent or anti-cancer therapy. Alternatively, the components may be administered at the same time.

The compositions and combination of agents used in the methods described herein may be administered as a single bolus dose, a dose over time such as an infusion, as in intravenous, subcutaneous, or transdermal administration, or in multiple dosages. If infusion is used, the combination may be infused for about 15 minutes to about 6 hours. In one embodiment, the infusion may occur for the duration of length of the apheresis. Additionally, the compositions or combinations may be administered once daily for multiple days including from 1 to 4 days.

It also is conceivable that more than one administration of either the compound or the other therapy will be desired. Various combinations may be employed, where a compound of the present disclosure is “A,” and the other compound or therapy is “B,” as exemplified below:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

In other embodiments, the compositions or methods used herein may be administered with an anti-cancer therapy such as those described below. The methods or compositions described herein may be used in conjunction with standard methods or variations as practiced by a person of ordinary skill in the art. These anti-cancer agents may be administered prior to and/or concomitant with the compositions or methods described herein. Some non-limiting examples of anti-cancer therapies which may be used herein include chemotherapy, immunotherapy, or radiotherapy.

Tumor-Specific Drug Release

In some embodiments, the disclosed compositions can be modified to incorporate a labile or cleavable linker (see e.g., Example 4). This strategy can be used for more tumor-specific drug release and is based on an enzyme labile linker. For example, in pancreatic tumors and tumor microenvironment, a protease called cathepsin B is highly upregulated. A cathepsin B-sensitive dipeptide linker can be used to deliver gemcitabine. Upon the CCR2 binding mediated internalization of CuNCs-ECL1 i-Gem, gemcitabine will be specifically released within tumor cells and tumor-associated macrophages and it will be much more stable in serum for efficient clearance to reduce toxicity concerns. It is believed that this is the first pairing of a labile linker with the presently disclosed CCR2 targeting strategy for improved treatment outcomes.

Other tumor-specific cleavable linkers (e.g., protease-cleavable peptide) can be as described in Weidle et al. Cancer Genomics & Proteomics March 2014, 11(2) 67-79.

Detecting Ccr2/Ccl2 (Mcp-1) Associated Disease, Disorders, or Conditions

As described herein, the present disclosure provides for methods of detecting or imaging CCR2 receptors or evaluating or monitoring a CCR2 associated disease, disorder, or condition. CCR2 receptors are upregulated in CCR2 associated disease, disorders, or conditions.

Because of the relationship between CCL2 and CCR2, a CCR2 associated disease can also be a disease associated with CCL2 (MCP-1).

Chemokines, or chemotactic cytokines, are small heparin-binding proteins that constitute a large family of peptides (60-100 amino acids) structurally related to cytokines, whose main function is to regulate cell trafficking, particularly that of immune cells, and thus are of relevance to this BRTC application.

Chemokines can be classified into four subfamilies on the basis of the number and location of the cysteine residues at the N-terminus of the molecule and are named CXC, CC, CX3C, and C. They initiate their cellular effects via interaction with a specific G protein-coupled receptor. Monocyte chemo-attractant protein chemokines are secreted by a wide variety of cell types under a range of inflammatory conditions such as atherosclerosis, neurodegenerative disease, and various forms of cancer. One of the most prominent of these is monocyte chemoattractant protein-1 (MCP-1), now called CCL2, which significantly regulates migration and infiltration of monocytes to the site of inflammation, predominantly through CC-chemokine receptor 2 (CCR2). In the case of the monocyte subsets mentioned above CD16-/Ly6Chi pro-inflammatory monocytes exhibit high CCR2 expression, whereas the CD16+/Ly6Clo low-inflammatory monocytes do not. This interplay and its impact on monocyte trafficking and tissue inflammation really highlight the importance of CCR2 imaging to identify the critical pro-inflammatory monocyte subset as well as potentially track its migration from hematopoietic sites to sites of inflammation in both pre-clinical and clinical research.

CCR2 Associated Diseases, Disorders, or Conditions

A CCR2 associated disease can be any disease, disorder, or condition in which CCR2 is involved, the disease disorder, or condition is associated with CCR2 or a CCR2 mediated syndrome. CCR2 is implicated in many cancers, atherosclerosis, prostate cancer, lung transplantation, and lung injury, for example. CCR2 associated diseases can be an inflammatory diseases or cancer. For example, a CCR2 associated disease can be an inflammatory disease, metabolic disease (e.g., type II diabetes), necrosis, atherosclerosis, cancer (e.g., prostate cancer), multiple sclerosis, atheroma, monocytic leukemia, kidney diseases (e.g., glomerulonephritis), Hamman-Rich syndrome, endometriosis, rheumatoid arthritis, bronchiolitis, asthma, systemic lupus erythematosus, inflammatory bowel diseases (e.g., colitis), alveolitis, restinosis, brain trauma, psoriasis, idiopathic pulmonary fibrosis, transplant arteriosclerosis, vascular permeability and attraction of immune cells during metastasis, a number of different neurological disorders, autoimmune disease, obesity, multiple sclerosis, rheumatoid arthritis, pain, or pulmonary fibrosis.

As another example, the CCR2 associated disease, disorder, or condition can be an ophthalmic disorder, uveitis, atherosclerosis, abdominal aortic aneurysm, rheumatoid arthritis, psoriasis, psoriatic arthritis, atopic dermatitis, multiple sclerosis, Crohn's Disease, ulcerative colitis, nephritis, organ allograft rejection, fibroid lung, renal insufficiency, diabetes and diabetic complications, diabetic nephropathy, diabetic retinopathy, diabetic retinitis, diabetic microangiopathy, tuberculosis, chronic obstructive pulmonary disease, sarcoidosis, invasive staphyloccocia, inflammation after cataract surgery, allergic rhinitis, acute or chronic sinusitis allergic conjunctivitis, chronic urticaria, asthma, allergic asthma, periodontal diseases, periodonitis, gingivitis, gum disease, diastolic cardiomyopathies, cardiac infarction, myocarditis, chronic heart failure, angiostenosis, restenosis, reperfusion disorders, glomerulonephritis, solid tumors and cancers, chronic lymphocytic leukemia, chronic myelocytic leukemia, multiple myeloma, malignant myeloma, Hodgkin's disease, and carcinomas of the bladder, breast, cervix, colon, head and neck, lung, prostate, or stomach.

As another example, the CCR2 mediated syndrome, disorder, or disease can be age-related macular degeneration or retinal degeneration. As another example, the CCR2 mediated syndrome, disorder, or disease can be a cardiovascular disease, especially ischemia of lower members or of the heart, or atherogenesis. As another example, the CCR2 mediated syndrome, disorder, or disease can be pain, in particular peripheral pain, such as pain from the sciatic nerve. As another example, the CCR2 mediated syndrome, disorder, or disease can be acute and chronic lung diseases-acute lung injury, primary graft dysfunction (PGD) (a reperfusion injury after transplant), COPD, asthma, pulmonary fibrosis, bronchiolitis obliterans syndrome, and fungal pneumonia.

CCL2 is also associated with the neuroinflammatory processes that take place in various diseases of the central nervous system (CNS), which are characterized by neuronal degeneration. CCL2 expression in glial cells is increased in epilepsy, brain ischemia Alzheimer's disease experimental autoimmune encephalomyelitis (EAE), and traumatic brain injury.

As another example, CCR2 has been shown to be associated with idiopathic anterior uveitis; HIV-1; Cd3zeta deficiency; cytomegalovirus retinitis; rhinoscleroma; secondary progressive multiple sclerosis; lipid pneumonia; rheumatoid arthritis; or macular degeneration, age-related, 1.

As another example, CCL2 has been shown to be associated with neural tube defects; vangl1-related neural tube defect; HIV-1; Mycobacterium tuberculosis, susceptibility to Mycobacterium tuberculosis, protection against, included; proliferative glomerulonephritis; arthritis; rheumatoid arthritis; herpes simplex virus keratitis; anthracosis; crescentic glomerulonephritis; peritonitis; acute cystitis; arteriosclerosis; xanthogranulomatous pyelonephritis; mast-cell leukemia; psoriasis; trypanosomiasis; retinal vasculitis; diabetic macular edema; mesangial proliferative glomerulonephritis; Chagas disease; demyelinating disease; renal fibrosis; cerebral aneurysms; denture stomatitis; Kawasaki disease; verruciform xanthoma of skin; interstitial lung disease; severe acute respiratory syndrome; diabetic angiopathy; Erdheim-Chester disease; pulmonary alveolar proteinosis; uveitis; extrapulmonary tuberculosis; encephalitis; pneumonia; endometriosis; carotid artery disease; pneumoconiosis; retinal vein occlusion; abdominal aortic aneurysm; viral meningitis; glomerulonephritis; idiopathic interstitial pneumonia; nephrosclerosis; acute proliferative glomerulonephritis; viral encephalitis; pulmonary sarcoidosis; post-thrombotic syndrome; vascular disease; alcoholic hepatitis; papillary conjunctivitis; hyperhomocysteinemia; scleritis; radiculopathy; pulmonary fibrosis; lipoid nephrosis; pleural tuberculosis; autoinflammation, lipodystrophy, and dermatosis syndrome; pleurisy; complex regional pain syndrome; pyelonephritis; endocervicitis; leptospirosis; microvascular complications of diabetes 1; dengue shock syndrome; peripheral artery disease; chorioamnionitis; silicosis; pelvic inflammatory disease; vitreoretinopathy, neovascular inflammatory disease; purulent labyrinthitis; stachybotrys chartarum; transient cerebral ischemia; neuritis; keratitis; tuberculous meningitis; nonspecific interstitial pneumonia; limb ischemia; secondary progressive multiple sclerosis; retinal vascular occlusion; Israeli tick typhus; bacteriuria; pulmonary fibrosis, idiopathic; stromal keratitis; bone cancer; sarcoidosis 1; malaria; ureteral disease; coronary artery aneurysm; lung disease; macular holes; urinary tract obstruction; extrinsic cardiomyopathy; periodontitis; systemic lupus erythematosus; vasculitis; ariboflavinosis; eye disease; meningitis; artery disease; cystitis; central nervous system disease; macular degeneration, age-related, 1; obesity; multiple sclerosis, disease progression, modifier of; diabetes mellitus, noninsulin-dependent; urinary system disease; endometrial stromal sarcoma; myocardial infarction; degeneration of macula and posterior pole; overnutrition; respiratory system disease; acquired metabolic disease; or bone inflammation disease.

Chemokines (e.g., MCP-1/CCL2)

The present disclosure provides for an imaging probe available for CCR2 detection. As described herein, the CCR2 imaging specificity and sensitivity have been well characterized in pre-clinical studies.

Chemokines, or chemotactic cytokines, are small heparin-binding proteins that constitute a large family of peptides (60-100 amino acids) structurally related to cytokines, whose main function is to regulate cell trafficking, particularly that of immune cells, and thus are of relevance to this BRTC application. Chemokines can be classified into four subfamilies on the basis of the number and location of the cysteine residues at the N-terminus of the molecule and are named CXC, CC, CX3C, and C. They initiate their cellular effects via interaction with a specific G protein-coupled receptor. Monocyte chemo-attractant protein chemokines are secreted by a wide variety of cell types under a range of inflammatory conditions such as atherosclerosis, neurodegenerative disease and various forms of cancer. One of the most prominent of these is monocyte chemoattractant protein-1 (MCP-1), now called CCL2, which significantly regulates migration and infiltration of monocytes to the site of inflammation, predominantly through CC-chemokine receptor 2 (CCR2). In the case of the monocyte subsets mentioned above CD16-/Ly6C^{hi} pro-inflammatory monocytes exhibit high CCR2 expression, whereas the CD16+/Ly6C^{lo} low-inflammatory monocytes do not. This interplay and its impact on monocyte trafficking and tissue inflammation really highlight the importance of CCR2 imaging to identify the critical pro-inflammatory monocyte subset as well as potentially track its migration from hematopoietic sites to sites of inflammation in both pre-clinical and clinical research.

CCR2 directs monocytes and other immune cell recruitment in the lung. A major role for the CCL2/CCR2 pair is the recruitment of inflammatory monocytes from the bone marrow and regulation of macrophage, dendritic and T cells maturation. In response to CCL2, CCR2+ monocytes adhere to the vascular endothelial surface and migrate into tissue, along chemotactic gradients.

Inflammatory monocytes (mouse Ly6C^{hi} Ly6G^{lo}, human CD14⁺CD16⁻) serve as precursors for classical macrophages and conventional DCs. CCR2⁺ monocytes also provide a secondary source of proinflammatory modulators, such as tumor necrosis factor-α, interleukin-1β and matrix metalloproteinases, contributing to lung injury. Although inflammatory monocytes are essential early responders, excessive or prolonged recruitment impairs resolution of inflammation and propagates disease progression.

Formulation

The agents and compositions described herein can be formulated by any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating, preventing, or reversing cancer or tumor growth (e.g., in a CCR2-associated cancer, a CCR2-expressing tumor) in a subject in need of administration of a therapeutically effective amount of a CCR2-targeting composition (e.g., an anti-cancer and/or imaging agent), so as to image, treat, or deliver therapy to a cancer or tumor site.

Methods described herein are generally performed on a subject in need thereof. A subject in need of the therapeutic methods described herein can be a subject having, diagnosed with, suspected of having, or at risk for developing cancer. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a CCR2-targeting composition is, for example, an amount that would cause the desired therapeutic effect in a subject while minimizing undesired side effects. In various embodiments, an effective amount of a CCR2-targeting composition described herein can substantially prevent or inhibit cancer proliferation or tumor growth, slow the progress of cancer proliferation or tumor growth, or limit the development of cancer or tumor growth.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a CCR2-targeting composition can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to substantially prevent or inhibit cancer proliferation or tumor growth, slow the progress of cancer proliferation or tumor growth, or limit the development of cancer or tumor growth.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a CCR2-targeting composition can occur as a single event or over a time course of treatment. For example, a CCR2-targeting composition can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for cancer.

A CCR2-targeting composition can be administered simultaneously or sequentially with another agent, such as a chemotherapeutic agent, an immunotherapy, a checkpoint inhibitor, an antibiotic, an anti-inflammatory, or another agent. For example, a CCR2-targeting composition can be administered simultaneously with another agent, such as a chemotherapeutic agent, an immunotherapy, a checkpoint inhibitor, an antibiotic, or an anti-inflammatory. Simultaneous administration can occur through administration of separate compositions, each containing one or more of a CCR2-targeting composition, a chemotherapeutic agent, an immunotherapy, a checkpoint inhibitor, an antibiotic, an anti-inflammatory, or another agent. Simultaneous administration can occur through administration of one composition containing two or more of a CCR2-targeting composition, a chemotherapeutic agent, an immunotherapy, a checkpoint inhibitor, an antibiotic, an anti-inflammatory, or another agent. A CCR2-targeting composition can be administered sequentially with a chemotherapeutic agent, an immunotherapy, a checkpoint inhibitor, an antibiotic, an anti-inflammatory, or another agent. For example, a CCR2-targeting composition can be administered before or after administration of an antibiotic, an anti-inflammatory, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Screening

Also provided are methods for screening for compounds and compositions for treating cancer using the platform as described herein.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

KITs

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to a CCR2-targeting composition (e.g., anti-cancer/imaging agents), nanoparticles, radionuclides, radiolabeled particles, immunotherapies, CCR2 binding peptides, or chemotherapeutic agents. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: CCR2-Targeting Ultrasmall Copper Nanoparticles for Pet-Guided Delivery of Gemcitabine for Pancreatic Ductal Adenocarcinoma

Abstract

Pancreatic ductal adenocarcinoma (PDAC) is a deadly malignancy with dire prognosis due to aggressive biology, lack of effective tools for early detection, and limited treatment options. Detection of PDAC using conventional radiographic imaging is limited by the dense, hypovascular stromal component and relatively scarce neoplastic cells within the tumor microenvironment (TME). The C-C motif chemokine 2 (CCL2) and its cognate receptor CCR2 (CCL2/CCR2) axis is critical in fostering and maintaining this kind of TME by recruiting immunosuppressive myeloid cells such as the tumor-associated macrophages, thereby presenting an opportunity to exploit this axis for both diagnostic and therapeutic purposes. As disclosed herein, CCR2-targeting ultrasmall copper nanoparticles (CuNPs) were engineered as nano-vehicles not only for targeted PET imaging by intrinsic radiolabeling with ⁶⁴Cu but also for loading and delivery of chemotherapy drug gemcitabine to PDAC. This ⁶⁴Cu radiolabeled nano-vehicle allowed sensitive and accurate detection of PDAC malignancy in autochthonous genetically engineered mouse models. The ultrasmall CuNPs showed efficient renal clearance, favorable pharmacokinetics and minimal in vivo toxicity. Systemic administration of gemcitabine loaded CuNPs significantly inhibited the growth of PDAC tumors in a syngeneic xenograft mouse model and prolonged survival. These CCR2-targeted ultrasmall nanoparticles offer a promising image-guided therapeutic agent and show great potential for translation.

Introduction

Pancreatic ductal adenocarcinoma (PDAC) is one of the deadliest forms of human cancer and is a growing health problem in the United States. The median survival of patients with advanced-staged disease is less than one year, and the 5-year overall survival rate is just ˜9%. Surgical resection with adjuvant systemic chemotherapy currently offers the best chance for cure but success is limited to 10-15% of patients who are diagnosed at early stage, with the majority of patients succumbing eventually to disease relapse. Most patients are diagnosed at locally advanced or metastatic stages that preclude the potential for successful tumor removal. Combination chemotherapies can prolong survival but are not curative. Factors contributing to the high mortality of PDAC include lack of accurate biomarkers for early diagnosis, rapid metastatic spread to the lymphatic system and distant organs, and dearth of effective therapies. Conventional cytotoxic treatments, such as single or multi-agent chemotherapy regimens using gemcitabine (Gem), or multidrug regimens, have been evaluated in clinical trials, however, long term outcome remains poor.

Resistance to chemotherapy remains a daunting medical challenge in the treatment of PDAC. The tumor microenvironment (TME) of PDAC is characterized by a desmoplastic stroma featuring an abundance of fibroblasts, leukocytes, endothelial cells, and extracellular matrix proteins. This unique stroma poses physical and biological barrier to drug delivery. Notably, infiltrative leukocytes within the TME are mainly composed of bone-marrow-derived myeloid cells, including tumor-associated macrophages, which not only stifle the effect of chemotherapy and anti-tumor immunity but also promote tumor growth and metastasis. The chemokine ligand type 2 (CCL2, also called monocyte chemoattractant protein-1, MCP-1) and its receptor, chemokine receptor type 2 (CCR2), form a signaling axis that plays a key role in the recruitment of CCR2+ inflammatory monocytes from the bone marrow to the tumors, where they become immunosuppressive tumor-associated macrophages. The CCL2/CCR2 axis has been implicated in the pathogenesis of several disease processes including inflammation, atherosclerosis, and tumor growth and metastasis. Importantly, pre-clinical and phase I/II clinical studies have revealed that when coupled with CCR2 inhibition, therapeutic response to chemotherapy could be improved. However, these studies utilize small molecule inhibitors against CCR2, which are typically cytostatic and may become ineffective upon emergence of CCR2-independent adaptive response, underscoring the need to improve on the efficacy of CCR2-targeting strategies.

In the past decade, nanotechnology has led to advances in cancer therapy by delivering chemotherapeutic drugs directly to targeted tumor sites, prolonging drug release, and enhancing drug metabolism. To date, a number of nanoparticles have been developed for PDAC imaging or delivery of therapeutic drugs. Many nanoparticles showed encouraging results via the enhanced permeability and retention (EPR) effect despite the stromal barriers within TME of mice. Some nanoparticles had been used in PDAC patients although the treatment efficiency needs significant improvement. To further improve the diagnosis accuracy and treatment efficiency, the targeting ultrasmall nanoparticles have drawn significant interest as an emerging platform due to accurate tumor detection, favorable in vivo pharmacokinetics, rapid renal clearance, and potential for translational cancer theranostics. Previously, it has been reported that in vivo biodegradable copper nanoparticles intrinsically radiolabeled with ⁶⁴Cu (⁶⁴Cu-CuNPs) can be used for sensitive and accurate PET imaging of triple negative breast cancer. In contrast to other drug delivery systems, the accessibility of material makes the synthesis straightforward. The rapid pharmacokinetics enables low background for enhanced contrast. The intrinsic ⁶⁴Cu radiolabeling and high specific activity ensured imaging accuracy and sensitivity.

Disclosed herein is a CCR2 imaging agent using ECL1 i peptide demonstrated to specifically target monocytes and macrophages in multiple animal models. Ultrasmall ⁶⁴Cu-CuNPs were reconstructed with ECL1i to target tumor stroma, and gemcitabine (⁶⁴Cu-CuNPs-ECL1 i-Gem) was loaded for PET guided drug delivery into PDAC tumors. Specificity and sensitivity for targeted PET imaging were assessed in two genetically engineered pancreatic cancer mouse models. Treatment efficacy in a syngeneic xenograft mouse model and related in vivo toxicity were also assessed.

Results and Discussion

Synthesis of the Ultrasmall Copper Nanoparticles

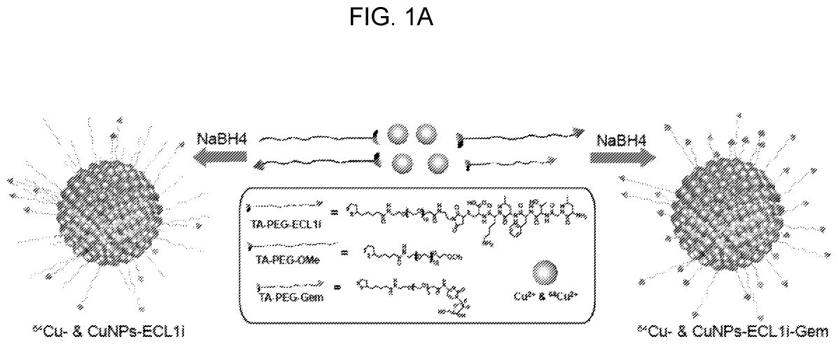

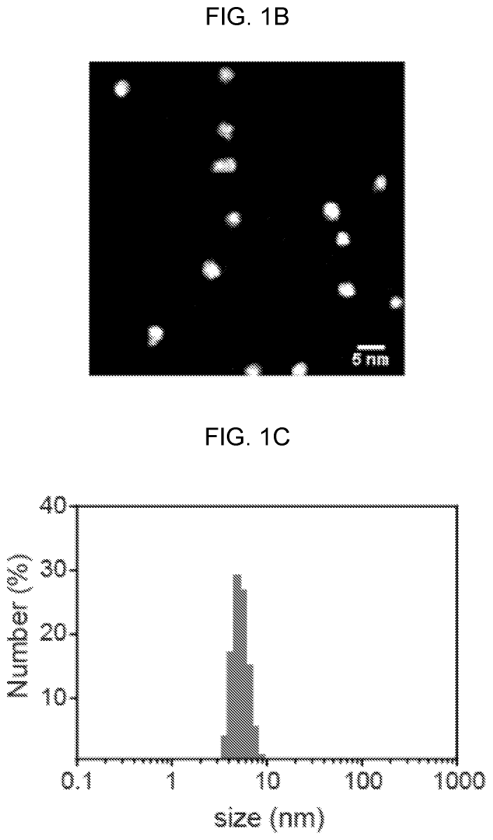

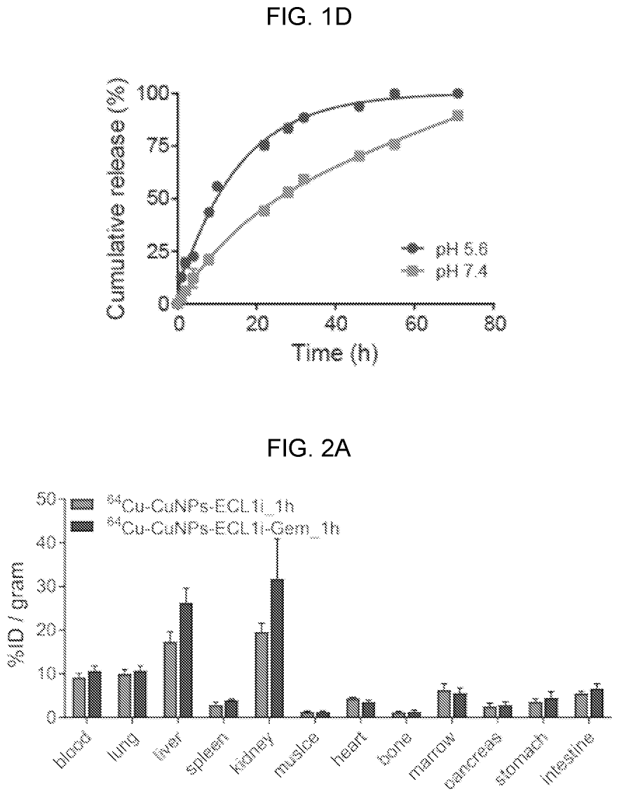

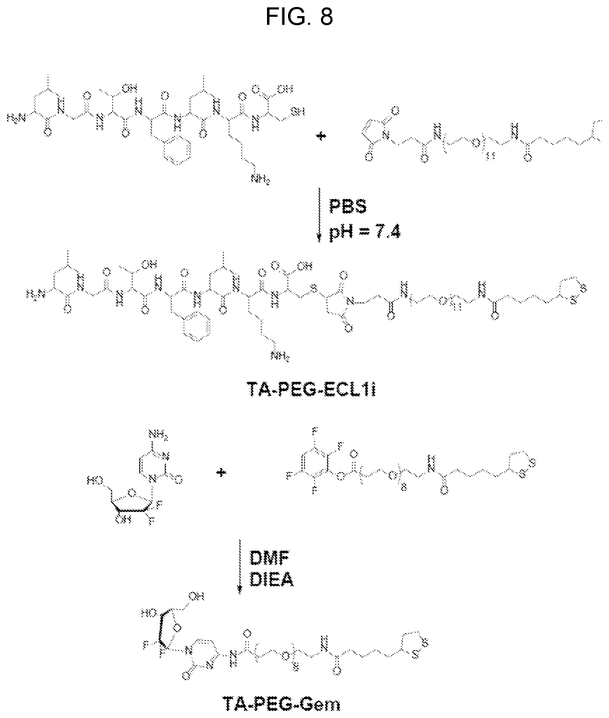

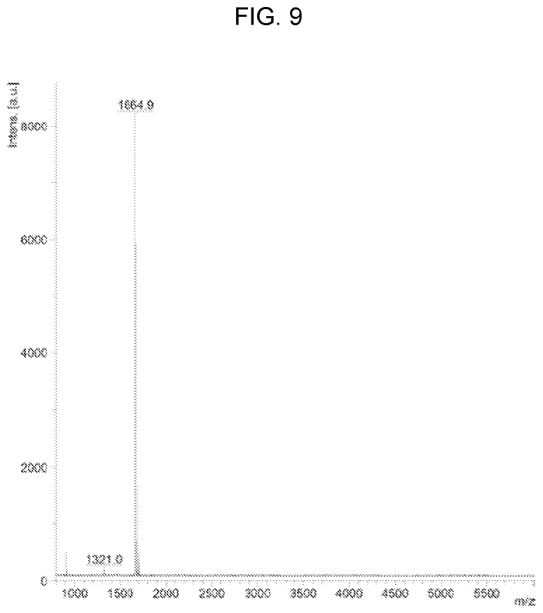

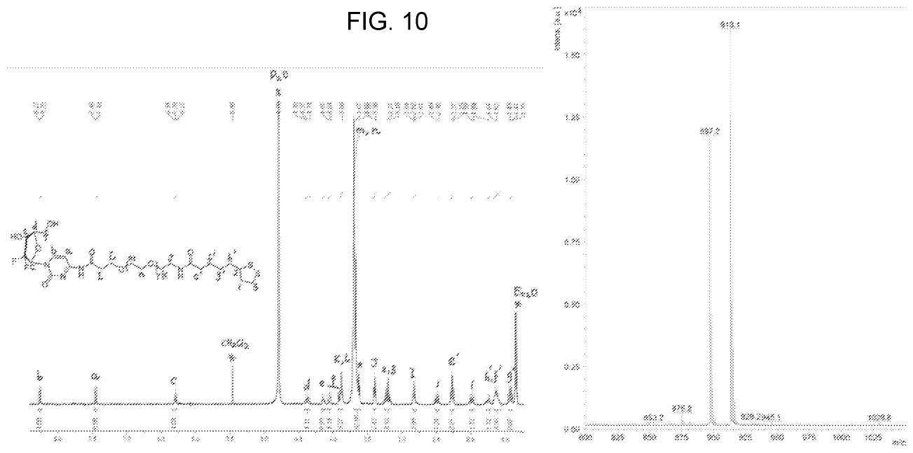

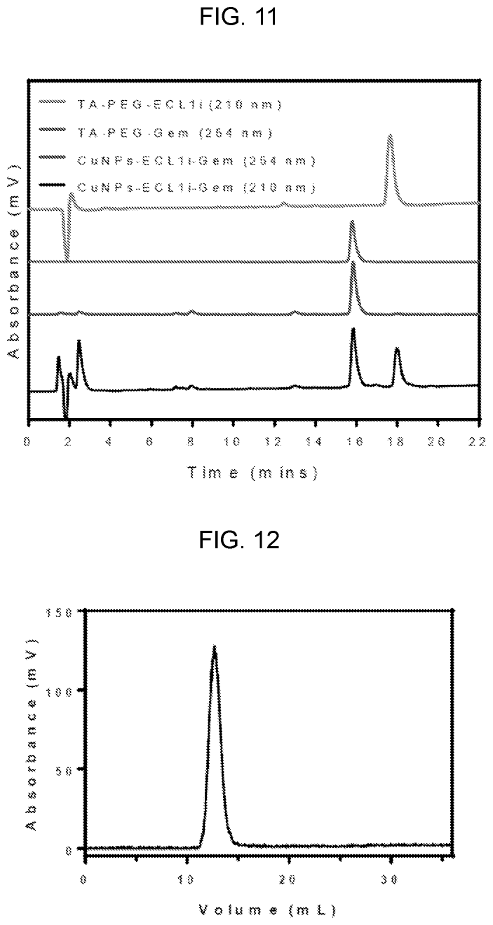

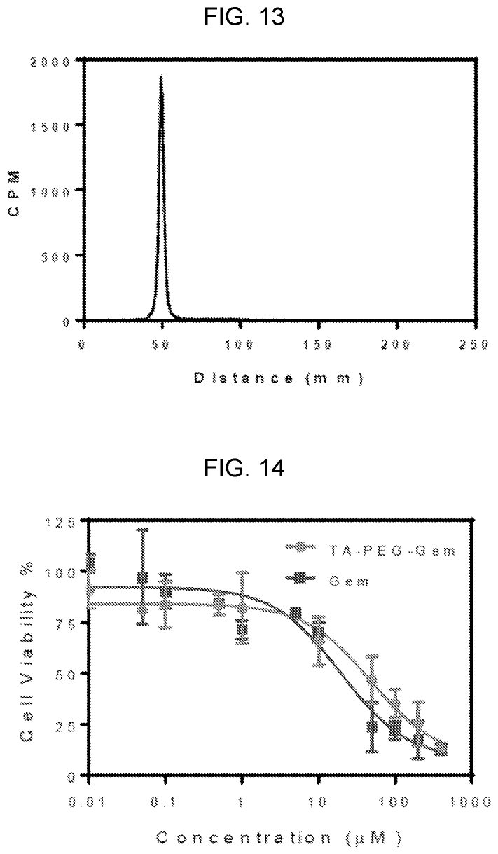

The synthesis of ⁶⁴Cu radiolabeled CCR2-targeting CuNPs (⁶⁴Cu-CuNPs-ECL1i) was modified from previous procedures (see e.g., FIG. 1A) with high yield and radiolabeling specific activity (see e.g., FIG. 1 and FIG. 8-FIG. 14). Scanning transmission electron microscopy (STEM) image of CuNPs-ECL1i showed uniform size with an average diameter of 2.7±0.2 nm (see e.g., FIG. 1B). Dynamic light scattering (DLS) measurement demonstrated a renal-clearable hydrodynamic diameter (5.3±0.1 nm, see e.g., FIG. 1C) and neutral surface charge (ξ-potential: −3.7±0.7 mV). Previously, it has been demonstrated that there are approximately 180 TA-PEGs on the surface of CuNPs. Based on the optimized targeting strategy disclosed herein, the synthesis was started with a 1:2 molar ratio (TA-PEG-ECL1 i: TA-PEG-OMe) for CuNP surface conjugation. High performance liquid chromatography (HPLC) and inductively coupled plasma mass spectrometry (ICP-MS) measurement determined there were approximately 60±1.3 copies of TA-PEG-ECL1i on each CuNP, consistent with previous work (see e.g., TABLE 1 and FIG. 11).

TABLE 1
Characterization of CuNPs.

| | | Zeta | | |
|---|---|---|---|---|
| | Hydrodynamic | potential | TA-PEG- | TA-PEG- |
| | diameter (nm) | (mV) | ECL1i (/NP) | Gem (/NP) |
| CuNPs | 5.4 ± 0.2 | −14.7 ± 0.8 | — | — |
| (CuNPs-NT) | | | | |
| CuNPs- | 5.3 ± 0.1 | −3.7 ± 0.7 | 60 ± 1.3 | — |
| ECL1i | | | | |
| CuNPs- | 4.9 ± 0.3 | −4.8 ± 2.4 | 53.0 ± 7.0 | 134.9 ± 6.7 |
| ECL1i-Gem | | | | |

Fast protein liquid chromatography (FPLC) analysis demonstrated the integrity and chemical purity of CuNPs-ECL1i after synthesis (see e.g., FIG. 12).

Gemcitabine loaded nanoparticles (CuNPs-ECL1 i-Gem) were synthesized following the same protocol using TA-PEG-Gem: TA-PEG-ECL1i at 2:1 molar ratio. The ξ-potential and hydrodynamic diameter of CuNPs-ECL1 i-Gem were −4.8±2.4 mV and 4.9±0.3 nm, respectively, demonstrating a minimal effect of Gem loading on CuNP surface charge and size. Quantitative measurement showed there were 134.9±6.7 copies of Gem and 53.0±7.0 copies of TA-PEG-ECL1 i per nanoparticle, with a loading efficiency of 81.0±4.1% and 63.3±8.4%, respectively. Gemcitabine is known to undergo rapid deamination in blood to become the inactive metabolite 2′,2′-difluorodeoxyuridine. In vitro cytotoxicity assay showed that TA-PEG modified gemcitabine (TA-PEG-Gem) had comparable IC₅₀ value (48.8 μM) to that of gemcitabine (19.7 μM) (see e.g., FIG. 14). Thus, this modification strategy and covalent conjugation of the prodrug onto CuNPs not only improved drug stability but also prolonged circulation half-life for enhanced drug delivery and treatment outcome. Moreover, the in vitro release profiles showed gradual release of TA-PEG-Gem in both physiological and acidic conditions, which was similar with the dissolution kinetics of the ultrasmall CuNPs, revealing the drug release was attributed to the dissolution of the nanoparticles (see e.g., FIG. 1D). Therefore, upon reaching the tumor in vivo, acidic conditions within tumor microenvironment would lead to the gradual dissolution of CuNPs to Cu(II) and subsequent release of gemcitabine within the tumors for improved treatment efficacy. With this strategy, the CuNPs-ECL1i-Gem were expected to accumulate in the tumoral regions on account of CCR2 targeting and release drug on-site for enhanced treatment effect.

Pharmacokinetics of ⁶⁴Cu-CuNPs-ECL1i and ⁶⁴Cu-CuNPs-ECL1i-Gem

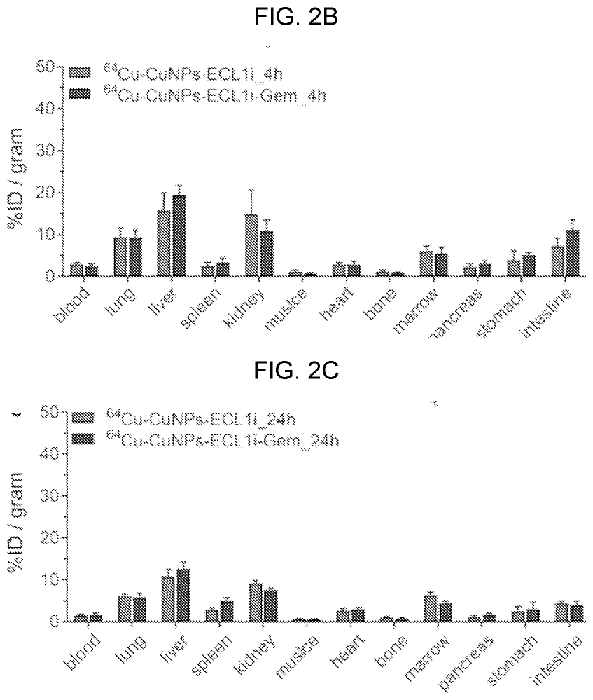

In vivo pharmacokinetics of ⁶⁴Cu-CuNPs-ECL1 i and ⁶⁴Cu-CuNPs-ECL1i-Gem were evaluated by biodistribution studies in C57BL/6 mice. As shown in FIG. 2, both ⁶⁴Cu-CuNPs-ECL1i and ⁶⁴Cu-CuNPs-ECL1 i-Gem showed rapid blood clearance with comparable half-lives (t_{1/2}=1.27 h for ⁶⁴Cu-CuNPs-ECL1i and t_{1/2}=0.81 h for ⁶⁴Cu-CuNPs-ECL1 i-Gem), consistent with previous reports. At 4 h post injection, 66.8% of ⁶⁴Cu-CuNPs-ECL1 i and 77.4% of ⁶⁴Cu-CuNPs-ECL1 i-Gem were cleared from blood compared to the data acquired at 1 h. At 24 h, the blood retentions of both nanoparticles were less than 2.0 percent injected dose per gram (% ID/g), providing a low background for enhanced tumor uptake contrast.

In contrast to high mononuclear phagocyte system (MPS) retention of metal nanoparticles with sizes more than 10 nm, the retentions of ⁶⁴Cu-CuNPs-ECL1 i and ⁶⁴Cu-CuNPs-ECL1 i-Gem in both the liver and spleen were significantly lower due to their ultrasmall sizes. Compared to ⁶⁴Cu-CuNPs-ECL1i, the accumulations of ⁶⁴Cu-CuNPs-ECL1 i-Gem were slightly higher at all three time points during the 24 h study. This is mainly due to the conjugation of relatively more hydrophobic gemcitabine comparing to hydrophilic ECL1 i peptide on the surface of CuNPs, which led to a more hydrophobic surface and related hepatic and splenic accumulations as reported by others.

Both nanoparticles revealed efficient renal clearance during the 24 h study. Due to their smaller size, the kidney clearance of ⁶⁴Cu-CuNPs-ECL1i-Gem (31.8±9.2% ID/g) at 1 h post injection was approximately 60% higher than that of ⁶⁴Cu-CuNPs-ECL1i (19.7±1.9% ID/g). This rapid urinary clearance of the drug loaded nanoparticles is particularly helpful to minimize potential toxicity caused by the retention of nanostructures in non-targeted organs. In other major organs, the two nanoparticles demonstrated comparable localization during the 24 h study, in agreement with previous reports.

Assessment of Tumor Targeting Specificity of ⁶⁴Cu-CuNPs-ECL1i In Vitro and in Autochthonous PDAC Mouse Models

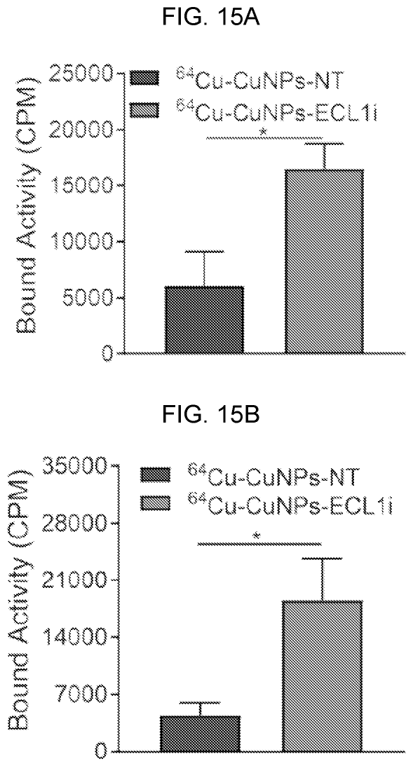

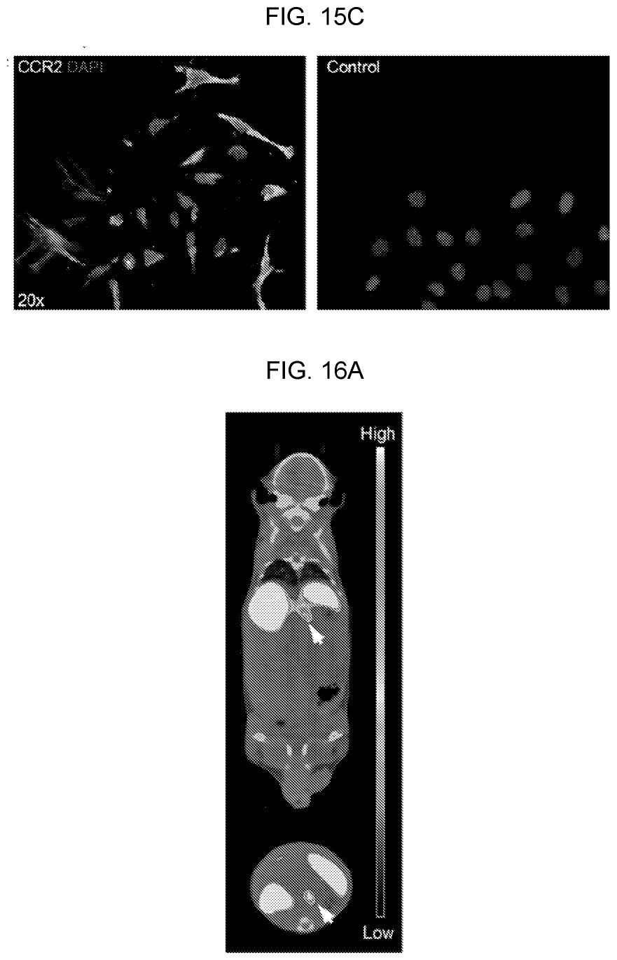

To estimate the CCR2 targeting specificity of ⁶⁴Cu-CuNPs-ECL1 i, the in vitro cell uptake was first assessed using both a PDAC tumor cells (KI, PDAC cells derived from KRASG12D/INK4A deficient mice) and a human monocytic THP-1 cell line. As shown in FIG. 15, after incubation at 0° C. for 30 mins, the cell uptake of ⁶⁴Cu-CuNPs-ECL1 i was nearly 4-fold and 3-fold as much as that of the non-targeted ⁶⁴Cu-CuNPs (⁶⁴Cu-CuNPs-NT) in KI and THP-1 cells (p<0.05, n=3), respectively, suggesting the CCR2 targeting specificity of ⁶⁴Cu-CuNPs-ECL1i.

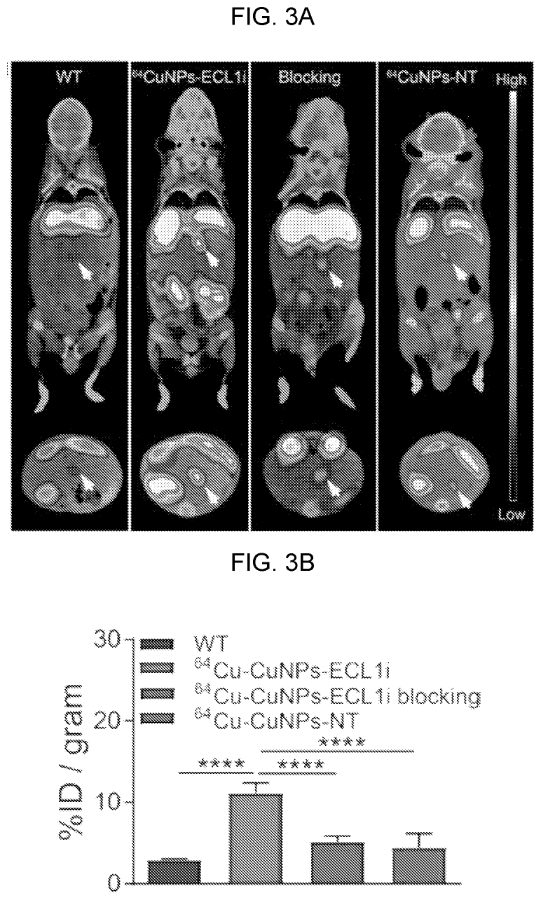

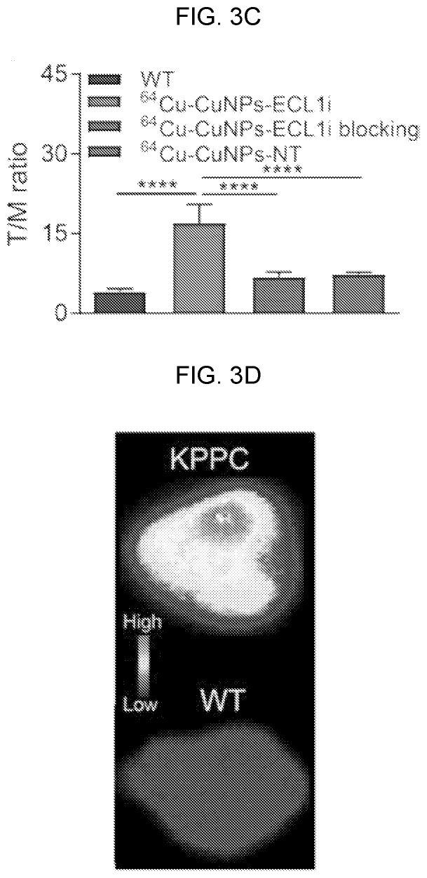

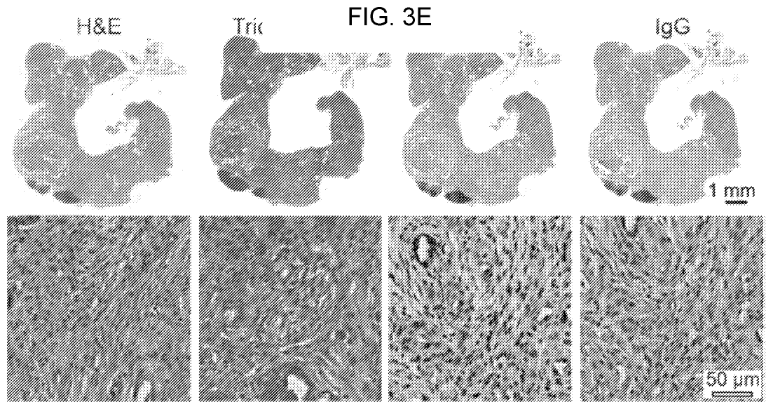

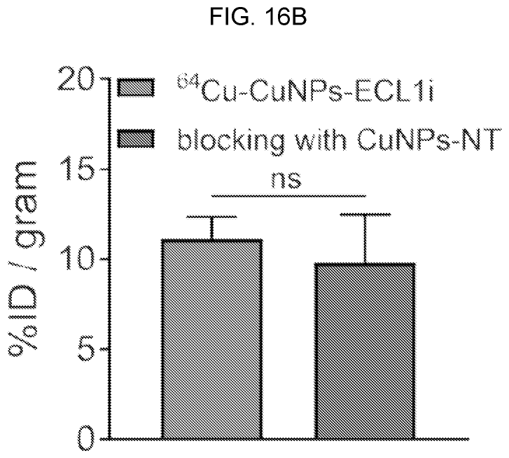

Next, the CCR2 targeting specificity of ⁶⁴Cu-CuNPs-ECL1i was assessed with PET/CT in two genetically engineered mouse models including the p48-CRE; LSL-KRasG12D/wt; p53flox/flox (KPPC) and p48-CRE; LSL-KrasG12D/wt; p53flox/wt (KPC) mice. These models are characterized by a stromal-rich TME and aggressive biology that highly mimics human PDAC, and are the most commonly used models in PDAC research. In these models, neoplastic progression occurs spontaneously, along with desmoplastic changes at different paces, with invasive PDAC foci typically detected in 6-week-old KPPC mice and ˜15-week-old KPC mice. As shown in FIG. 3, the 24 h PET image revealed increased retention of ⁶⁴Cu-CuNPs-ECL1 i in the tumors of 7-9-week-old KPPC mice in contrast to the minimal non-specific retention in those imaged with ⁶⁴Cu-CuNPs-NT. Quantitative uptake analysis showed that ⁶⁴Cu-CuNPs-ECL1 i tumor uptake (11.16±1.22% ID/g, n=5) was approximately 3-fold as much as that acquired with ⁶⁴Cu-CuNPs-NT (4.43±1.74% ID/g, p<0.0001, n=4). In wild type (WT) control littermate mice, the accumulation of ⁶⁴Cu-CuNPs-ECL1i (2.90±0.14% ID/g, p<0.0001, n=4) in pancreas was 4 times less of that obtained in KPPC mice. Competitive receptor blocking using a 50-fold excess of non-radioactive CuNPs-ECL1 i significantly decreased tumor accumulation of ⁶⁴Cu-CuNPs-ECL1i (5.14±0.70% ID/g, p<0.0001, n=5) by more than 50%. Moreover, blocking study using 50-fold excess of CuNPs-NT showed little effect on 64Cu-CuNPs-ECL1i tumor uptake (9.84±2.67% ID/g, n=4, p>0.05, see e.g., FIG. 16). All these data demonstrated the CCR2 targeting specificity of ⁶⁴Cu-CuNPs-ECL1 i in KPPC tumors.

Due to the significant tumor uptake and low non-specific accumulation at the surrounding tissue, the tumor/muscle (T/M) contrast ratio of ⁶⁴Cu-CuNPs-ECL1 i in KPPC tumors was 17.03±3.43 (n=5) at 24 h, highlighting its sensitivity for tumor localization. The significantly decreased T/M ratio in the blocked KPPC tumors (6.83±0.95, p<0.001, n=5) further confirmed the tumor targeting specificity of ⁶⁴Cu-CuNPs-ECL1 i.

Following PET imaging, tumors were immediately collected and fixed for autoradiography and histopathological characterization. As shown in FIG. 3D, ex vivo autoradiography of ⁶⁴Cu-CuNPs-ECL1 i in KPPC tumor showed intense and heterogeneous distribution across the tumor while minimal retention was determined in the pancreas of WT littermates, which further confirmed the PET data. Hematoxylin and eosin (H&E) and trichrome staining showed extensive fibrotic regions in the KPPC tumor (see e.g., FIG. 3E), representing the desmoplastic stromal formation during malignancy development and progression. 3,3′-diaminobenzidine (DAB) immunostaining showed elevated expression of CCR2 across the tumor while the control IgG staining had little signal, which further supported the PET imaging (see e.g., FIG. 3E).

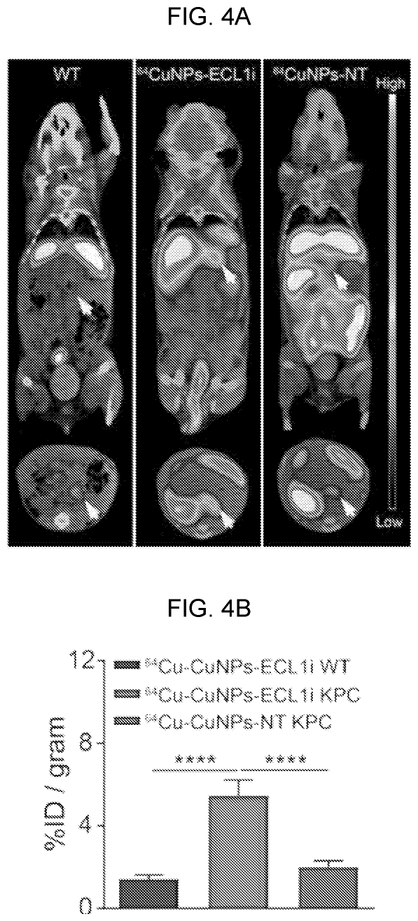

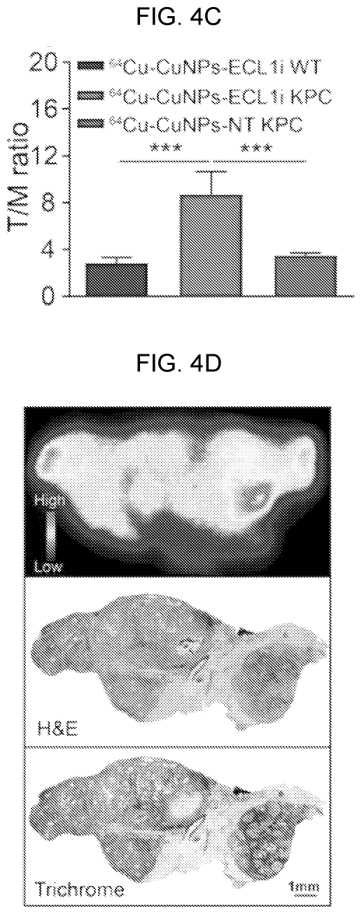

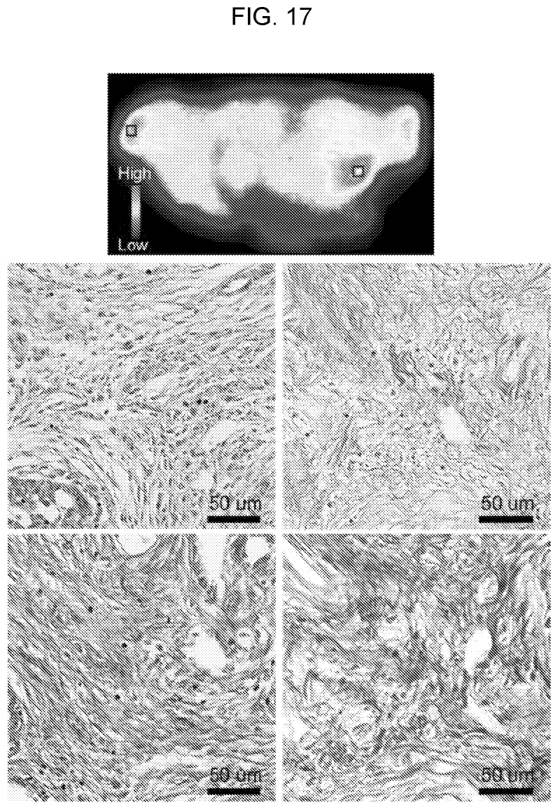

The PDAC targeting efficiency of ⁶⁴Cu-CuNPs-ECL1 i was further assessed in a more chronic tumor model using approximately 22-23-week-old KPC mice, when PDAC tumors are fully developed and recapitulate many pathological features of human PDAC, including progressive development of fibrosis and extensive accumulation of macrophages. Similar to the results obtained in the KPPC model, targeting ⁶⁴Cu-CuNPs-ECL1i showed a strong PET signal in the KPC tumor versus little retention in WT littermates, and non-targeting ⁶⁴Cu-CuNPs-NT showed low non-specific tumor accumulation suggesting targeting specificity (see e.g., FIG. 4A). Quantitative analysis showed the uptake of ⁶⁴Cu-CuNPs-ECL1i at 5.47±0.75% ID/g (n=5) in the KPC tumors, which was statistically higher than the non-targeting ⁶⁴Cu-CuNPs-NT in KPC mice (2.01±0.29% ID/g, p<0.0001, n=4) and ⁶⁴Cu-CuNPs-ECL1 i in WT mice (1.43±0.20% ID/g, p<0.0001, n=4) (see e.g., FIG. 4B). The T/M ratio of ⁶⁴Cu-CuNPs-ECL1 i (8.71±1.94, n=5) was more than twice as that acquired with ⁶⁴Cu-CuNPs-NT (3.51±0.21, p<0.001, n=5) in the KPC tumors, reconfirming the advantage of targeted imaging. Whole tissue H&E and trichrome staining revealed significant tumor progression and fibrosis. Ex vivo autoradiography acquired immediately following PET imaging showed accumulation of ⁶⁴Cu-CuNPs-ECL1 i in the fibrotic region, indicating binding to the tumor stroma (see e.g., FIG. 17). Interestingly, tumor uptake and the T/M ratio of ⁶⁴Cu-CuNPs-ECL1 i in the KPC mice were much lower than those in the KPPC mice, suggesting the advanced stroma in KPC tumors which limits the access and binding to CCR2+ cells. In contrast to a ¹¹¹In-labeled anti-claudin-4 monoclonal antibody, though the tumor accumulations were comparable, ⁶⁴Cu-CuNPs-ECL1i revealed higher contrast, reasonably due to the faster systemic clearance of the ultrasmall nanoparticles generating a low background comparing to the extended blood retention of antibodies.

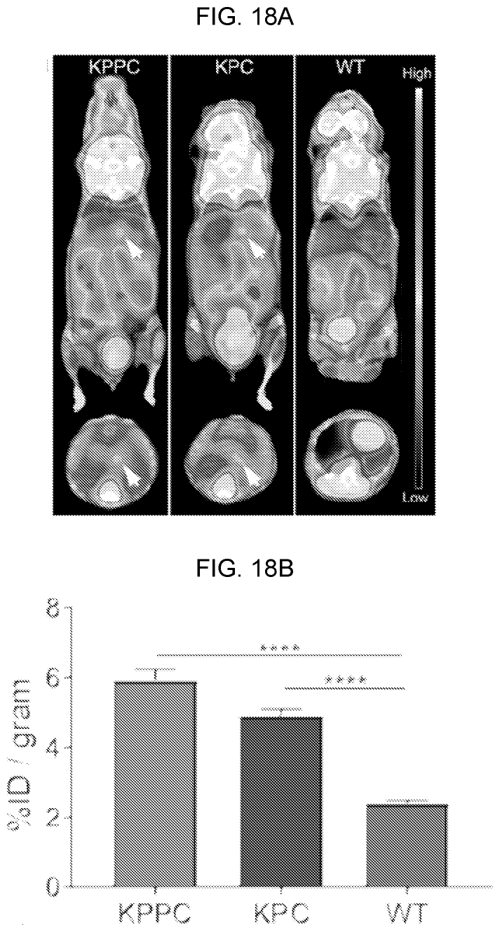

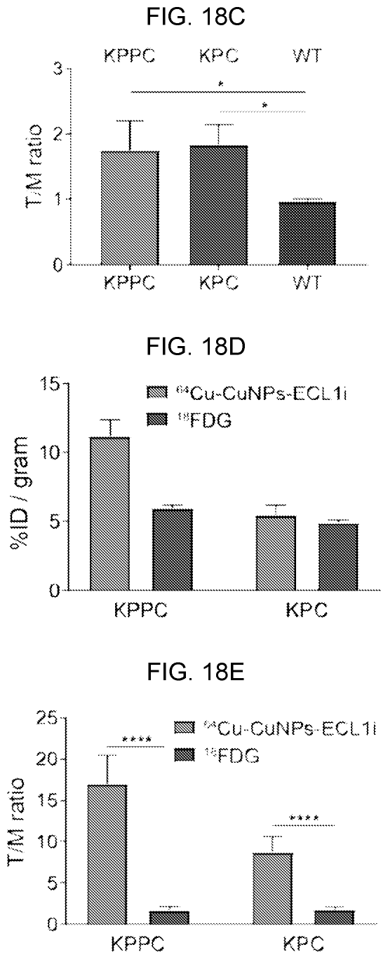

⁶⁴Cu-CuNPs-ECL1i imaging was also compared to ¹⁶F-FDG PET/CT in both KPPC and KPC mouse models. Though ¹⁶F-FDG signals were detected in both models (see e.g., FIG. 18), quantitative analysis showed ¹⁶F-FDG tumor uptake of 5.94±0.17% ID/g in KPPC mice which was approximately half the value of ⁶⁴Cu-CuNPs-ECL1 i (p<0.0001, n=4-5/group). In the KPC model, ¹⁸F-FDG uptake (4.91±0.10% ID/g, n=4-5/group) was comparable with that of ⁶⁴Cu-CuNPs-ECL1 i. However, the T/M ratios of ¹⁶F-FDG in KPPC (1.75±0.26, p<0.0001) and KPC (1.84±0.17, p<0.0001) mice were 9 and 3 times lower than those acquired with ⁶⁴Cu-CuNPs-ECL1 i, respectively. This was likely due to the non-specific nature of ¹⁶F-FDG uptake, leading to high background and low contrast ratio, which further highlighted the significance of CCR2 targeting ⁶⁴Cu-CuNPs-ECL1i for PDAC diagnosis.

CCR2 Targeted Gem Delivery Using CuNPs-ECL1i-Gem as a Novel Therapeutic Agent in PDAC

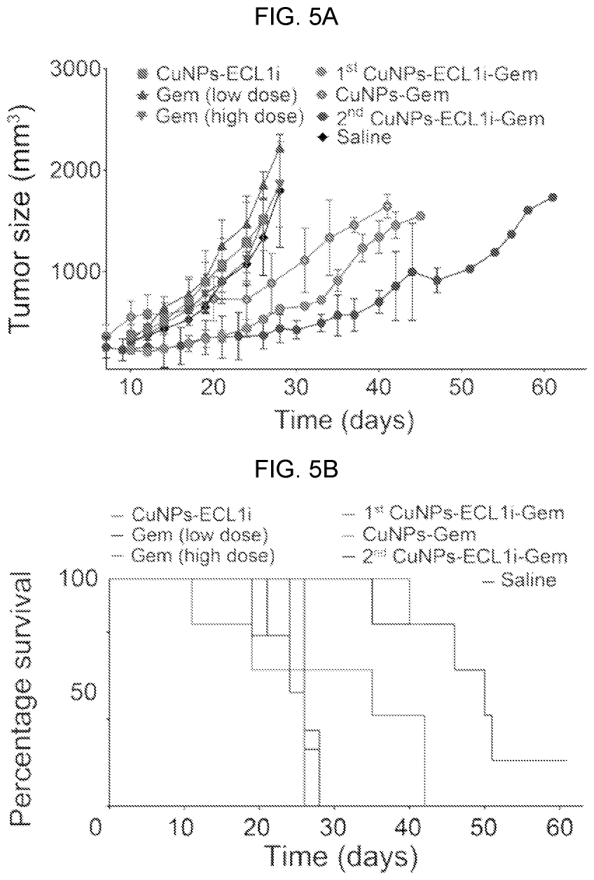

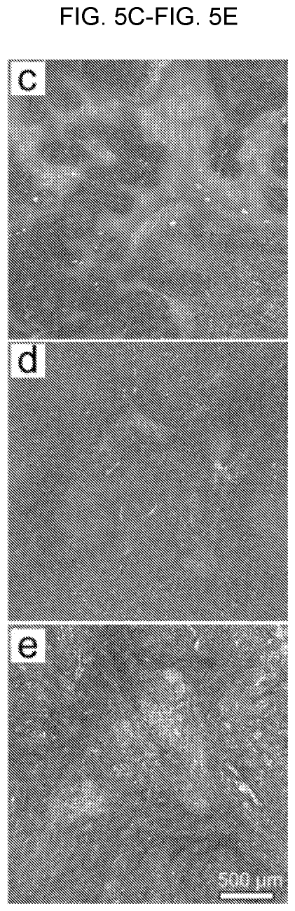

Gemcitabine has been widely used for advanced and metastatic PDAC treatment, alone and combined with other agents. However, these therapeutic regimens have not dramatically improved long-term outcomes. The suppressive immune cells, including CCR2+ myeloid cells in the TME, may contribute to chemoresistance. A recent clinical trial showed that pharmacologic CCR2 inhibition augments the clinical response to FOLFIRINOX chemotherapy to enable successful surgical resection for patients with borderline-resectable or locally advanced PDAC, at least in part by alleviating the immunosuppressive TME. On this premise, it may be hypothesized that elimination, rather than inhibiting CCR2 receptor, of CCR2+ myeloid cells using CuNPs-ECL1 i-Gem may be a more effective approach, which as disclosed herein was tested in a syngeneic immunocompetent xenograft (KI) model. This model was used because KPC cells have the propensity to ulcerate when grown subcutaneously, precluding prolonged treatment. As shown in FIG. 5A, the implanted tumors of KI mice treated with CuNPs-ECL1i, low dose Gem (7 mg/kg) via I.V. administration, and high dose Gem (100 mg/kg) via I.P. administration at day 10 post tumor implantation did not show any treatment benefit compared to mice injected with saline. The negligible treatment effect of CuNPs-ECL1 i suggested the Gem delivery vehicle itself had no therapeutic effect given the concerns of potential copper cytotoxicity. Thus, no survival benefit was determined in these groups compared to saline (median survival: CuNPs-ECL1i=25 days, Gem low dose=25 days; Gem high dose=26 days; saline=25 days) (see e.g., FIG. 5B).

Based on the PET imaging results, treatment efficiency was first assessed using the CCR2 targeted CuNPs-ECL1 i-Gem (Cu: 10 mg/kg, Gem loading: 3.4±0.8 mg/kg). As shown in FIG. 5A, the KI tumor growth was effectively suppressed as compared to the other control groups. At day 21, the tumor sizes of CuNPs-ECL1 i-Gem group (363.2±56.3 mm3, n=5) were approximately two times smaller than those measured in saline group (897.4±170.5 mm3, p<0.05, n=4). This was reasonably due to the targeted delivery of Gem via the CCR2 mediated tumor retention of CuNPs-ECL1 i-Gem abovementioned, which also significantly extended the median survival of KI mice to more than 42 days (see e.g., FIG. 5B).

In a repeated effort to further assess the treatment efficiency of CuNPs-ECL1 i-Gem, KI mice were treated with non-targeting CuNPs-Gem and CuNPs-ECL1 i-Gem at day 7 post tumor implantation. Though CuNPs-Gem showed tumor inhibition due to the low, non-specific delivery of Gem, the CCR2 targeting CuNPs-ECL1 i-Gem revealed substantial tumor inhibition with a statistical difference (p<0.01, n=5/group) in tumor size observed at day 33. Importantly, the effective tumor inhibition prolonged the median survival of KI mice treated with CuNPs-ECL1 i-Gem to 51 days compared to 35 days with CuNPs-Gem, highlighting the significance of targeted Gem delivery for PDAC treatment. Moreover, in contrast to the results obtained with CuNPs-ECL1 i-Gem treatment starting at day 10, the median survival was extended more than 20% (9 days), emphasizing the importance of early intervention. Histopathological characterization of the tumors treated with CuNPs-ECL1 i-Gem revealed a large necrotic region, in comparison, the tumors in mice receiving only Gem and saline had less necrotic levels, further confirming the effective tumor treatment by CuNPs-ECL1 i-Gem. The effective tumor growth inhibition of CuNPs-ECL1 i-Gem may not only be attributed to the targeted delivery of gemcitabine to tumor, but also the syngeneic effect of CCR2 targeting directed inhibition of tumor-associated macrophages recruitment.

CuNPs-ECL1i-Gem In Vivo Toxicity

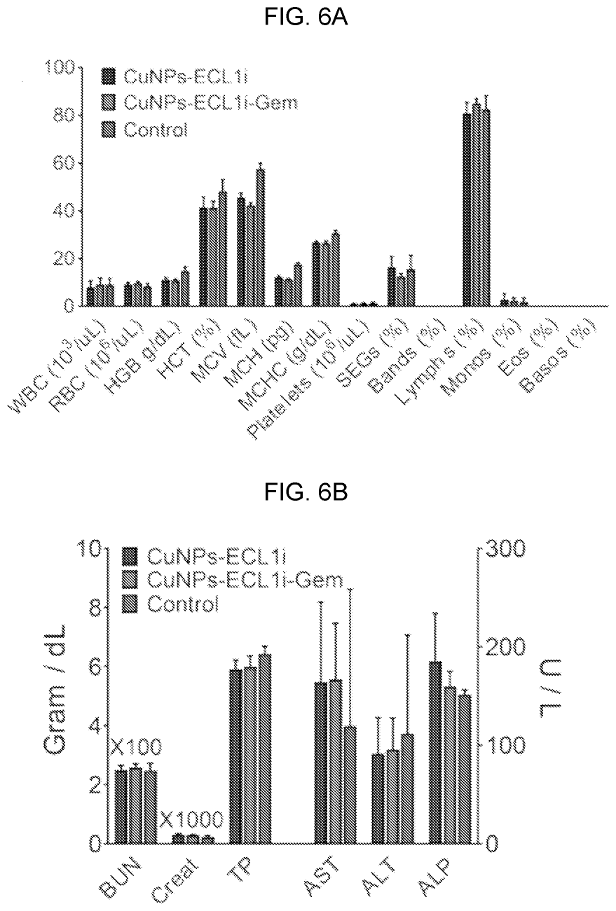

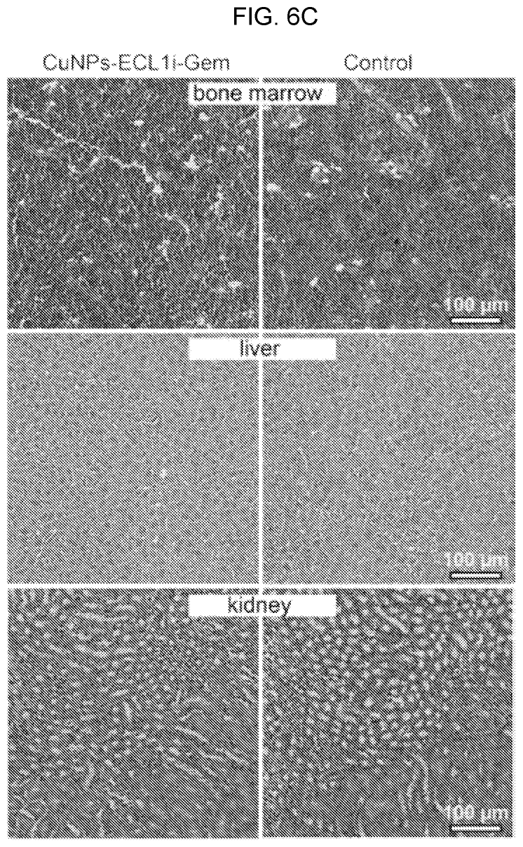

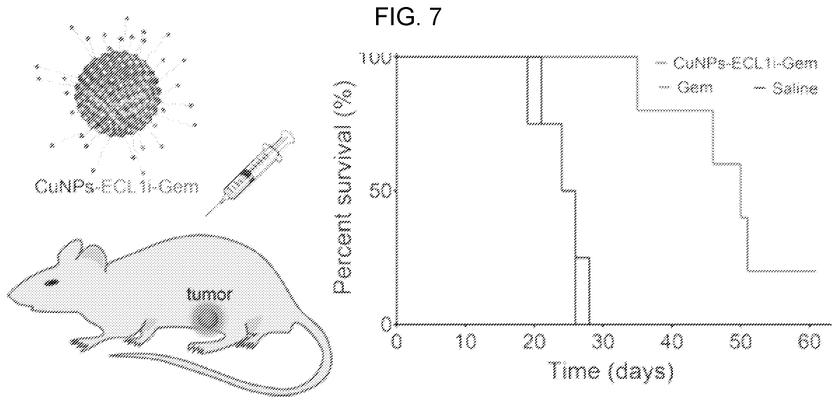

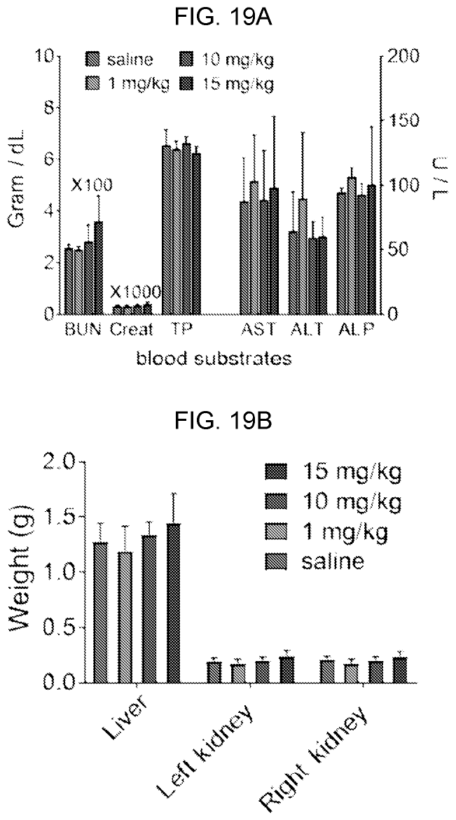

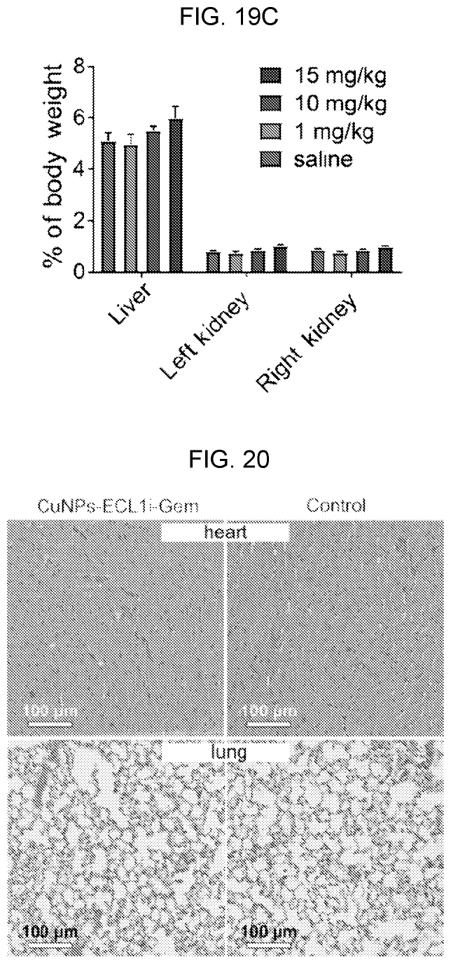

Cu is an essential element for bodies, but a surplus of Cu intake may cause adverse health problems. Therefore, the in vivo toxicity of the CuNP scaffolds was assessed in WT CD1 mice at dosages of 1, 10, 15, and 20 mg Cu/kg body weight via I.V. injection. The mice receiving 20 mg/kg CuNPs-NT died 2-3 days following administration, while the other groups of mice survived with no behavior changes, no hepatic or renal deficiencies as measured by serum, and no difference in liver and kidney weight as a percentage of body weight (see e.g., FIG. 19). Histopathologic examination showed no obvious lesions in major organs including lung, heart, liver, kidney, and bone marrow. Thus, 15 mg Cu/kg body weight was determined to be a safety threshold for one-time administration. Next, the cumulative toxicity of CuNPs-ECL1 i and CuNPs-ECL1 i-Gem (Cu mass=10 mg/kg) were determined in WT C57BL/6 mice following administration twice per week for 2 weeks. Complete blood count and white blood cell differential measurements, as well as serum biochemistries, validated that the dosage regimen had no adverse influence on hematopoietic, hepatic, and renal functions (see e.g., FIG. 6A and FIG. 6B). Gross necropsy and histopathologic examination showed no appreciable differences in bone marrow, liver, and kidney for CuNPs-ECL1i-Gem compared to the mice administrated with saline. Bone marrow samples from control and treated animals revealed no differences in cellularity or composition of the marrow. Microscopic examination revealed liver parenchyma with preserved architecture and no significant portal inflammation, steatosis or fibrosis in both control and treated groups. Likewise, kidney samples from control and treated animals revealed renal parenchyma with no significant glomerular sclerosis, tubular damage, inflammation or interstitial fibrosis (see e.g., FIG. 6C). Comparison of heart samples from control and treated animals revealed no inflammation or myocyte necrosis in either group while lung specimens revealed no significant acute or chronic inflammation or fibrosis (see e.g., FIG. 20).

The overall histological findings supported the safety of CuNPs-ECL1i-Gem for PDAC treatment in mice. This also highlighted the advantages of effective renal clearance and the degradable nature of CuNPs as previously reported. This rapid renal clearance led to low retention of unbound CuNPs-ECL1 i-Gem in major organs, reducing the long-term toxicity. The gradual dissolution of CuNPs could effectively decrease the toxicological effect of Cu for improved biocompatibility.

Conclusion

In summary, disclosed herein is a CCR2-targeting ultrasmall nanoparticle for PDAC imaging and therapy. The straightforward synthesis of ⁶⁴Cu-CuNPs-ECL1 i enabled high radiolabeling specific activity, scale-up and drug loading capability for accurate PET imaging and effective drug delivery. The ultrasmall nanostructure exhibited favorable pharmacokinetics and effective systemic clearance to minimize potential toxicity. In both KPPC and KPC mouse models, ⁶⁴Cu-CuNPs-ECL1 i demonstrated specific tumor detection and low nonspecific retention. The high T/M ratio of ⁶⁴Cu-CuNPs-ECL1i in comparison with ¹⁶F-FDG highlighted its potential for early and sensitive detection of PDAC malignancy. The CCR2 targeted therapy of CuNPs-ECL1 i-Gem led to substantial tumor necrosis, effective inhibition of tumor growth, and prolonged survival. Preliminary toxicity evaluations demonstrated the biocompatibility of both CuNPs-ECL1 i and CuNPs-ECL1 i-Gem for PDAC imaging and therapy.

The PDAC imaging was done with ⁶⁴CuNPs-ECL1 i while the treatments were with CuNPs-ECL1 i-Gem, based on the study design. Although there were no obvious differences in the physicochemical properties and in vivo pharmacokinetics between the two platforms, in future studies it may be desirable to investigate the theranostic potential of ⁶⁴Cu-CuNPs-ECL1 i-Gem for PDAC. Additionally, the current treatment regimen may benefit from further optimization such as dosage and frequency to enhance the therapeutic outcomes. It may also be desirable to study the treatment efficacy combining CuNPs-ECL1 i-Gem with other clinically used chemotherapeutic agents including paclitaxel or nab-paclitaxel for improved outcome.

Taken together, the CCR2-targeting nanoparticles disclosed herein demonstrated the potential for PDAC imaging guided therapy and warrant further investigation for future translation.

Experimental Section

Materials and Methods

Chemicals. Lipoamido-dPEG®8-TFP ester (TA-PEG-TFP ester), MAL-dPEG®11-lipoamide (TA-PEG-maleimide) and m-dPEG®12-Lipoamide (TA-PEG-OMe) were purchased from Quanta BioDesign, Ltd. and used as received. The CCR2 peptide (LGTFLKC) was synthesized by CPC Scientific (Sunnyvale, CA). The ⁶⁴Cu (half-life=12.7 h, β+=17%, β−=40%) was produced at the Washington University Cyclotron Facility. All other solvents and chemicals were purchased from Millipore-Sigma, TCI America, or Fisher Scientific and were used as received. Water with a resistivity of 18.2 MΩ cm was prepared using an E-Pure filtration system from Barnstead International (Dubuque, IA). ^{1H}NMR spectra were recorded on a 400 MHz NMR spectrometer (Varian/Agilent, Santa Clara, CA). Matrix assisted laser desorption/ionization time-of-flight (MALDI-TOF) data were collected using a Bruker AutoFlex Speed in a reflector mode with positive ion detection. JEOL JEM-2100F Field Emission Electron Microscope operating at an accelerating voltage of 200 kV was used for higher-resolution TEM imaging and scanning transmission electron microscopy (STEM) imaging. Samples were prepared by dropping a nanoparticle suspension onto an ultrathin carbon film supported by a lacey carbon film on a copper grid and air drying. DLS and ξ-potential measurements were conducted using Zetasizer Nano ZS from Malvern instruments. Radiochemical purity was determined by instant radio-thin layer chromatography (radio-TLC) (Bioscan). RP-HPLC analysis and preparation were conducted on an Agilent 1200 system with Phenomenex® Luna 5 μm 100 Å C18 LC column and Vydac 218TP™ 5 μm 300 Å C₁₈ semipreparative column, respectively. ICP-MS was performed on a PerkinElmer Elan DRCII instrument. FPLC was evaluated on AKTA FPLC system (GE Healthcare) with Superdex® 200 Increase 10/300 GL gel filtration column eluting by phosphate buffered saline (PBS). UV absorbance was obtained in a Victor 3 1420 multilabel counter (Perkin Elmer).

### Synthesis of TA-PEG-ECL1i

ECL1i peptide (50.0 mg, 64.0 μmol) and TA-PEG-maleimide (51.4 mg, 58.1 μmol) were mixed in 25 mL 0.1 M pH=7.0 phosphate buffer solution. After overnight stirring at R.T., TA-PEG-ECL1i was purified by RP-HPLC with H₂O/acetonitrile mobile phase and lyophilized to obtain a white powder (yield=55%). MALDI-TOF m/z: [M+H]+ Cal. for C₇₈H₁₂₉N₁₁O₂₄S₃H 1664.8; Found 1664.9, [M+Na]+ Cal. for C₇₅H₁₂₉N₁₁O₂₄S₃Na 1686.8; Found 1686.9, [M+K]+ Cal. for C₇₅H₁₂₉N₁₁O₂₄S₃K 1702.8; Found 1702.9.

### Synthesis of TA-PEG-Gem

Gemcitabine hydrochloride (50 mg, 0.17 mmol) and N,N-diisopropylethylamine (DIPEA, 29 μL, 0.17 mmol) were mixed in 200 μL anhydrous DMSO. After gemcitabine was dissolved, TA-PEG-TFP ester (130 mg, 0.17 mmol) in 200 μL anhydrous DMSO was added, followed by 29 μL DIPEA. The mixture was stirred overnight at 80° C. Next, the mixture was precipitated into diethyl ether twice, and washed with saturated NaHCO₃ and brine. The product was obtained as a pale yellow oil (yield=80%). ¹H NMR (D₂O, 400 MHz) δ (ppm): 8.26 (d, J=7.7 Hz, 1H), 7.45 (d, J=7.7 Hz, 1H), 6.29 (t, J=7.4 Hz, 1H), 4.42-4.33 (m, 1H), 4.15 (s, 1H), 4.06 (d, J=12.8 Hz, 1H), 3.89 (dd, J=12.8, 5.9 Hz, 3H), 3.70-3.60 (m, 30H), 3.39 (t, J=5.1 Hz, 2H), 3.25-3.16 (m, 3H), 2.82 (t, J=5.7 Hz, 2H), 2.49 (dd, J=12.9, 6.4 Hz, 1H), 2.28 (d, J=7.5 Hz, 2H), 1.98 (dd, J=12.9, 6.4 Hz, 1H), 1.73-1.63 (m, 4H), 1.42 (d, J=7.6 Hz, 2H). MALDI-TOF m/z: [M+Na]+ Cal. for C₃₆H₆₀N₄O₁₄S₂F₂Na 875.4; Found 875.4, [M+K]+ Cal. for C₃₆H₆₀N₄O₁₄S₂F₂K 913.3; Found 913.3.

### Synthesis of ⁶⁴Cu-CuNPs-ECL1i and ⁶⁴Cu-CuNPs-ECL1i-Gem

The synthesis of copper nanoparticles was modified from previous methods. Typically, CuCl₂ (376 μL, 10 mM), TA-PEG ligands (total 400 μL, 2.5 mM (⁶⁴Cu-CuNPs-ECL1 i used 1:2 molar ratio of TA-PEG-ECL1 i and TA-PEG-OMe, ⁶⁴Cu-CuNPs-ECL1 i-Gem used 1:2 molar ratio of TA-PEG-ECL1 i and TA-PEG-Gem), and ⁶⁴CuCl₂ (in 0.1 M NH4OAc, pH 5.5, ca. 18.5 MBq μL−1) were mixed in 2 mL water and stirred for 15 mins at RT. Then, sodium borohydride (450 μL, 20 mM) was added to the mixture with rapid stirring and stirred for another 25 mins. The prepared nanoparticles were centrifuged using a centrifugal filter unit (Amicon Ultra, 10 kDa NMWL) and washed 3 times with water. The scale up nanoparticles for treatment were filtered through 0.22 μm filter (Corning® 50 mL Tube Top Vacuum Filter System) prior to centrifugation. Radiochemical purity was determined by instant radio-thin layer chromatography (iTLC or radio-TLC) using glass microfiber chromatography paper impregnated with a silica gel (Agilent Technology) and 10% ammonium acetate and methanol (1:1 volume ratio) mixture as developing solution (Radio-TLC, BioScan).

Cell Culture

KI cells derived from KRASG12D/INK4A deficient mice, and THP1 cells were cultured in Dulbecco modified Eagle medium with L-glutamine (Mediatech, Manassas, VA) and 10% fetal bovine serum (Sigma, St. Louis, MO), gentamicin (50 μg/ml), and amphothericin B (0.25 μg/ml). All cells were cultured at 37° C., 5% C02, and 95% humidity.

Mouse Tumor Model

Conditional p48-CRE; LSL-KRas^{G12D/wt}; and p53^{flox/flox} 1-Cre strains were interbred to obtain KPPC and KPC animals on a mixed 129/SvJae/C57Bl/6 background. For the KI model, KI cell line (obtained from a PDX1-Cre/KRAS^{G12D}/INK4A^{flox/flox} mouse) was expanded and inoculated subcutaneously into syngeneic 8 week-old female FVBN/J mice. All studies were conducted in compliance with the institutional animal care and use committee (IACUC) guidelines of the Washington University.

Cytotoxicity Study

The KI Cells were seeded into 96-well plates at a density of 5000 cells per well. 24 hrs later, the medium was replaced by fresh medium containing TA-PEG-Gem and Gem at the indicated concentration (range, 0-500 μM), respectively. All experimental incubation conditions were performed at least in triplicate. Cell viability was determined after 3 days of continuous drug treatment using the WST-1 cell cytotoxicity assay (Roche, Basel, Switzerland) following the manufacturer's instruction. Briefly, 10 μl WST-1 reagent was added in each well followed by additional incubation for 4 h. The absorbance at 440 nm was measured using a microplate reader. IC50 was calculated using GraphPad Prism 7.03 software (GraphPad Software, San Diego, CA).

Cell Uptake Studies

10,000 THP1 or KI cells were incubated in the medium with 0.074 MBq ⁶⁴Cu-CuNPs-NT and ⁶⁴Cu-CuNPs-ECL1 i at 0° C. for 30 mins, respectively. Each group was performed in triplicate. The unbonded nanoparticles were removed by washing the cells with PBS for 4 times. The radioactivity was measured in a PerkinElmer 1480 automatic gamma counter and recorded as counts per minute (cpm).

Immunofluorescence and Microscopy

KI cells were incubated on coverslips (Chemglass Inc, CLS-1760-012) in 24 well plates for 24 h at 37° C. After being washed with phosphate-buffered solution (PBS) and fixed with 10% neutral-buffered formalin (Sigma, St. Louis, MO) for 15 mins at room temperature, the cells were blocked for 30 mins in 10% BSA (ThermoFisher, Waltham, MA). Then the cells were stained with mouse anti-CCR2 antibody (Abcom, Cambridge, MA, 1:100 dilution) or 10% BSA for 1 h at room temperature, washed by PBS three times, and incubated with donkey anti-mouse Cy5 antibody (Jackson ImmunoResearch, West Grove, PA, 1:300 dilution) and DAPI (ThermoFisher, 1:1000 dilution) for 1 h. The samples were visualized on macroscope camera (Leica, DC7000T) after putting the coverslip on the slide with mounting medium.

Biodistribution

All animal experiments were carried out in compliance with IACUC guidelines of the Washington University. Female wild type C57BL/6 mice (Charles River Laboratories, Wilmington, MA) were used for the biodistribution studies. The mice were anesthetized with inhaled isoflurane and were injected with about 370 kBq of ⁶⁴Cu-CuNPs-ECL1i or ⁶⁴CuNPs-ECL1i-Gem in 100 μL saline (APP pharmaceuticals, Schaumburg, IL) via the tail vein. At 1, 4, and 24 h post injection, the mice were re-anesthetized and euthanized by cervical dislocation (n=4/time point). Organs of interest were collected, weighed, and counted in a Beckman 8000 gamma counter (Beckman, Fullterton, CA). Standards were prepared and measured along with the samples to calculate percentage of the injected dose per gram of tissue (% ID/g). The mean blood half-lives of the two nanoparticles were calculated based on the blood retention data using a nonlinear regression analysis (Prism, version 7.03, Graphpad).

Micro-PET/CT Imaging

KPPC mice at 7-9-week-old, KPC mice at 22-23-week old, and their wild type littermates at the same age were anesthetized and injected with 3.7 MBq ⁶⁴Cu-CuNPs-ECL1i/⁶⁴Cu-CuNPs-NT in 100 μL saline via the tail vein. Small animal PET scans were performed on Inveon PET/CT system (Siemens, Malvern, PA) at 24 h post injection (60 min frame). The micro-PET images were corrected for attenuation, scatter, normalization, and camera dead time and co-registered with micro-CT images. All PET scanners were periodically cross-calibrated. Micro-PET images were reconstructed with the maximum a posteriori (MAP) algorithm and analyzed by Inveon Research Workplace. Tumor uptake was calculated in terms of the percent injected dose per gram (% ID/g) of tumor tissue in three-dimensional ROIs without the correction for partial volume effect. A competitive PET blocking study was done by co-injection of 50-fold non-radioactive CuNPs-ECL1i/CuNPs-NT.

Autoradiography

The mice were transcardially perfused and the tumors were collected and sliced immediately following PET/CT scan. The slices were covered by a phosphor-imaging film plate and exposed overnight prior to imaging with a GE Typhoon FLA 9500 Biomolecular Imager.

Cu Toxicity Study

8-9 weeks old CD1 mice were IV administrated with 1, 10, 15, and 20 mg/kg body weight of CuNPs-NT (n=5/group). Saline was injected as the control group. The mice receiving 20 mg/kg of body weight of CuNPs-NT died 2-3 days post treatment. One-week post administration, the mice in other groups were submitted to Division of Comparative Medicine (DCM) research animal diagnostic laboratory in Washington University in St. Louis for clinical pathologic and histopathologic evaluation. Immediately following euthanasia by CO₂ inhalation, blood was collected from animals by cardiocentesis. Hematology was performed on blood samples anticoagulated with EDTA using commercially supplied tubes (Microvette 100, Sarstedt AG, Numbrecht, Germany). The complete blood count was performed using the Hemavet 1700 Veterinary Multispecies Hematology System (Drew Scientific, Miami Lakes, FL). Blood smears of each sample were prepared, dried, fixed in methanol, and stained using Wright-Giemsa stain for microscopic evaluation. Serum measurements of blood urea nitrogen (BUN), creatinine, alanine aminotransferase (ALT), aspartate aminotransferase (AST), alkaline phosphatase, and total protein were determined using the Liasys 330 liquid reagent chemistry analyzer (AMS Diagnostics, Weston, FL). The animal was weighed. Kidneys and liver were collected, weighed, and fixed in 10% neutral buffered formalin. After fixation, the tissues were trimmed, paraffin-embedded, and 5-micron sections were prepared and stained with hematoxylin and eosin for standard histopathologic evaluation. 8-week-old male and female C57BL/6 mice were randomly treated with (1) CuNPs-ECL1i-Gem; (2) CuNPs-ECL1i; and (3) saline (n=5/group) for 2 successive weeks. The Cu mass was equivalent to 10 mg/kg of body weight. 3 days post treatment, all the mice were submitted to DCM research animal diagnostic laboratory for evaluation.

Treatment

A KI xenograft model was used to monitor tumor growth trends and treatment efficacy of CuNPs-ECL1i-Gem. 10 days after tumor implantation, mice were randomized into 5 groups (n=5/group): (1) CuNPs-ECL1i-Gem (Cu mass: 10 mg/kg body weight, Gem: 3.4±0.8 mg/kg body weight, I.V. injection, twice per week); (2) CuNPs-ECL1i (same CuNPs-NT dose as (1), I.V. injection, twice per week); (3) gemcitabine 7 mg/kg body weight (I.V. injection, twice per week); (4) gemcitabine 100 mg/kg body weight (I. P. injection, once per week); (5) saline (100 μL, I.V. injection, twice per week). For the 2nd treatment study, the mice started dose administration at 7 days post implantation. The dose regimens of CuNPs-NT-Gem and CuNPs-ECL1i-Gem were the same as the 1st treatment study. The Cu mass was limited to a dosage of 10 mg/kg for all nanoparticle groups, and the relative loaded gemcitabine dose was 3.4±0.8 mg/kg body weight. The low dose gemcitabine group was comparable, in terms of gemcitabine dose, to the dosing regimen of CuNPs-ECL1i-Gem. The high dose gemcitabine group was comparable with the standard human dosing regimen.70 Mouse weight and tumor volume were monitored 2-3 times per week. Tumor volume was calculated as (length×width2)/2. The treatment was continued for 6 successive weeks or until the mice died or were sacrificed and deemed as dead when their tumor volume reached over 2000 mm³. After treatment, the tumors were collected for H&E staining.

Immunohistochemistry of Tumor Tissues

Tumor serial sections (5-μm thick) were cut from Histochoice®-fixed (24 h), paraffin-embedded specimens. The sections were deparaffinized and rehydrated through a series of xylenes and graded alcohols and stained with hematoxylin and eosin to assess morphology of the tissues. Consecutive sections of rehydrated tumor underwent antigen retrieval pre-treatment (Diva Decloaker, 1×) as well as hydrogen peroxide quenching before being treated with blocking serum for 1 hour to prevent nonspecific binding (Vectastain; Vector Laboratories, Burlingame, CA). The sections were incubated in primary antibody overnight at 4C (anti-CCR2, 1:1000 in blocking serum, Novus Biologicals). After secondary antibody was applied (Vector Laboratories, Burlingame, CA), brown color development was achieved through a diaminobenzidine-based immunostaining. Digital images of the stained sections were obtained using both scanning light and light microscopes (Nanozoomer, Hamamatsu, and Leica).

Statistical Analysis

Statistical analysis was performed using GraphPad Prism. Group variation was described as mean±standard deviation. Differences among two groups were determined using the unpaired two-tailed Student's t-test. A P value <0.05 was considered to represent a significant difference.

Loading Capacity of CuNPs-ECL1i-Gem

After purification, the volume of CuNPs-ECL1 i-Gem solution was adjusted to 200-400 μL with water. Then, 10 μL of the solution was added into 40 μL 0.1 M HCl and the mixture was stirred at room temperature for 30 mins. After the CuNPs were fully dissolved, the clear solution was diluted to 100 μL for HPLC analysis (mobile phase: A: 99.9% water, 0.1% TFA; B: 99.9% acetonitrile, 0.1% TFA; column: Luna® 5 μm C18 100 A 150×4.6 mm; HPLC condition: gradient elution 20%-40% B for 20 mins, 1.5 mL/min; UV: DAD detector, 254 nm for TA-PEG-Gem, 210 nm for TA-PEG-ECL1i). The peaks at 15.7 min and 17.6 min were identified as TA-PEG-Gem and TA-PEG-ECL1i and performed integration, respectively. The loading amount of TA-PEG-Gem and TA-PEG-ECL1 i were calculated according to their standard calibration curves. Assume the copper nanoparticle is a quasi-sphere, the number of Cu atoms (NCu) is calculated as

$${N}_{Cu} = \frac{4 π {R}^{3}}{3 {V}_{Cu}} = {( \frac{R}{{r}_{Cu}} )}^{3} \approx 960$$

where R is the radius of the nanoparticle, which was obtained from STEM, and r_{Cu} is the radius of the Cu atom. The concentration of Cu nanoparticles was obtained from the concentration of Cu atoms divided by N_{Cu}, where the concentration of Cu atoms was measured by ICP-MS. Finally, the loading capacity of the TA-PEG ligands were calculated from the concentration of the ligand divided by the concentration of Cu nanoparticles, respectively.

### Example 2: CCR2 Targeting Directed Nanoimmunotherapy of Pancreatic Ductal Adenocarcinoma

As disclosed herein (see Example 1), CCR2 targeted CuNC-ECL1i-Gem is effective in inhibiting KI tumor growth compared to other treatment regimens. Next, the efficiency of CCR2 targeting directed nanoimmunotherapy using CuNC-ECL1i-Gem plus immune checkpoint inhibitor including anti-CTLA and anti-PD-1 antibodies, was tested in a KI mouse model. This is based on the hypothesis that CuNC-ECL1i-Gem may kill the CCR2+ monocytes/macrophages that are major component of pancreatic cancer stromal to change the tumor microenvironment (TME) from immunosuppressive to immunoresponsive, which may boost the efficacy of immunotherapy using anti-PD-1 and anti-CTLA-4 antibodies.

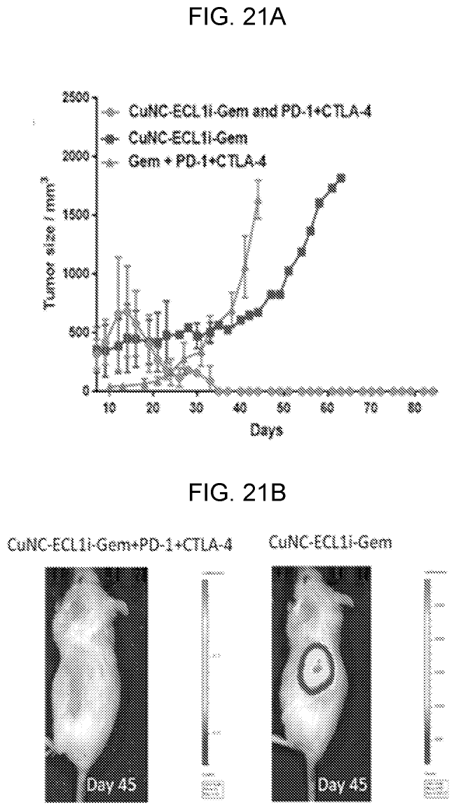

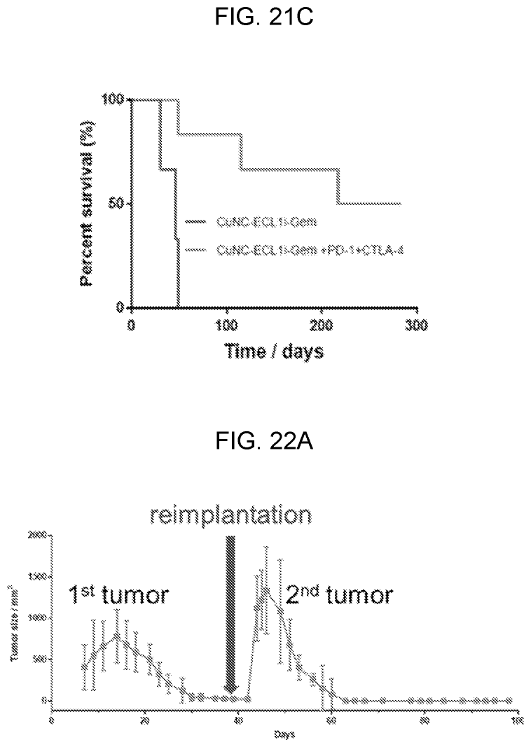

As shown below in FIG. 21, at day 7 post tumor implantation, the CuNC-ECL1i-Gem plus anti-CTLA-4 and anti-PD-1 antibodies (nanoimmunotherapy) group showed initial tumor growth until day 14 followed with rapid decrease of tumor sizes. At approximately 4 weeks post treatment, the CuNC-ECL1i-Gem and CTLA-4 and PD-1antibodies group (red curve) revealed complete removal of tumors in contrast to the continuous growth of tumors in CuNC-ECL1i-Gem treated group (blue curve) (see e.g., FIG. 21A). Additionally, using gemcitabine plus anti-PD-1 and anti-CTLA-4 treatment did not prevent the growth of tumor (green curve). Since the KI cells were transfected by luciferase, bioluminescent imaging was used to detect the change of tumor along the treatment. The luciferase signal in CuNC-ECL1i-Gem and antibodies treated mice was completely absent at day 45 post treatment while a strong signal was detected in CuNC-ECL1i-Gem treated mice (see e.g., FIG. 21B), which was consistent with the tumor measurement.

Importantly, the tumors in CuNC-ECL1i-Gem and PD-1+CTLA-4 group did not grow back since day 35 post implantation. At day 49, all the mice in CuNC-ECL1 i-Gem group died from tumors while the survival in nanoimmunotherapy group was still 50% at day 285 post implantation. Overall, these data confirmed that this strategy using CCR2 targeting directed nanoimmunotherapy is effective for PDAC treatment.

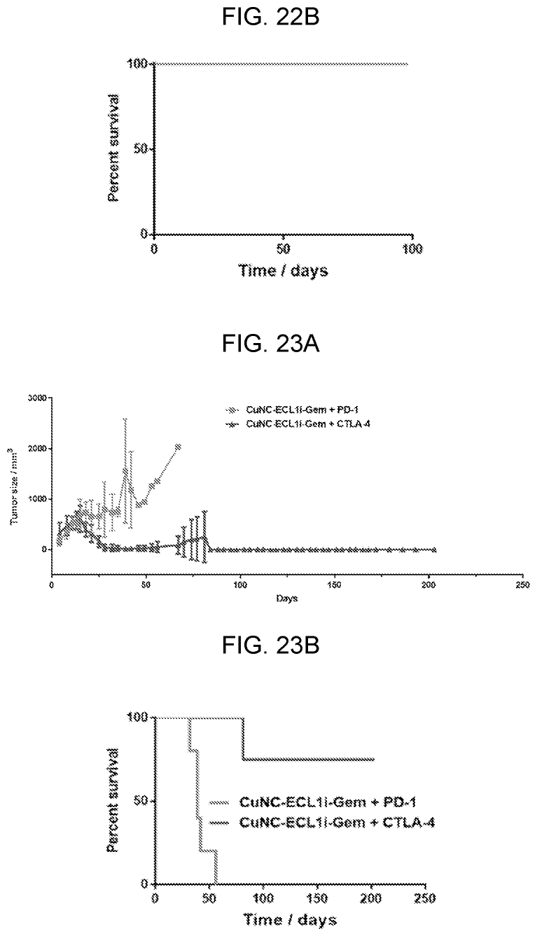

The treatment efficiency of CCR2 targeted nanoimmunotherapy was further tested by rechallenging the KI tumor mice after the tumors were erased. As shown in FIG. 22, following the treatment with CuNC-ECL1 i-Gem and PD-1+CTLA-4 starting at day 7 post implantation, KI tumors were completely removed by the nanoimmuotherapy at day 32. At day 38, same KI tumors were re-implanted on the contralateral side of thighs in these mice, followed by nanoimmunotherapy at day 12 post-challenge. As shown in FIG. 22A, the tumors totally disappeared again at day 63 post-treatment. During this 100 days study, all the tumor-bearing mice survived (see e.g., FIG. 22B).

Next, the contribution of each immune checkpoint inhibitor to the nanoimmunotherapy in KI tumors was assessed. As shown in FIG. 23, the combination of CuNC-ECL1 i-Gem+PD-1 showed similar treatment effect as CuNC-ECL1 i-Gem but CuNC-ECL1 i-Gem+CTLA-4 demonstrated comparable efficacy to that using both PD-1 and CTLA-4, suggesting that CTLA-4 is the effective immune checkpoint inhibitor in KI tumor treatment.

### Example 3: Favorable Intratumoral Immune Cells Changes Following Treatment with CuNPs-ECL1i-Gem

This example shows that following the CCR2 targeted nanotherapy, decreased tumor-associated macrophages (TAMs) and increased cytotoxic CD8+ T cells were observed.

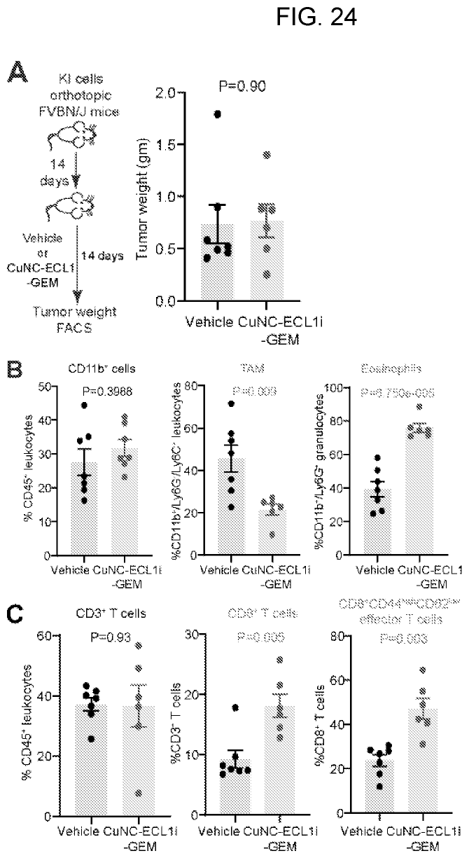

Immunocompetent FVBN/J mice inoculated with KRASG12D/INK4A pancreatic cancer cells in the pancreas were observed for 14 days followed by treatment with Vehicle (PBS) or CuNPs-ECL1 i-GEM by intravenous injections for two weeks. The pancreatic tumors were then harvested for flow cytometric analysis. In summary: CuNPs-ECL1 i-GEM treatment resulted in a significant decrease of tumor-associated macrophages, increase in eosinophils, CD8+ T cells and CD8+ effector T cells (see e.g., FIG. 24).

### Example 4: Tumor-Specific Drug Release

The objective of this example is to further optimize the construction of CCR2 targeted CuNCs to improve the stability of Gem loading during in vivo blood circulation and enhance its intracellular delivery for optimal treatment outcome.

Recently, protease-activated prodrugs have been an active area for site-specific delivery and improved stability. In pancreatic ductal adenocarcinoma (PDAC), the protease cathepsin B is highly upregulated in lysosomes and vesicles throughout the cytoplasma in both tumor cells and TAMs and closely associated with the progression and metastasis of PDAC. Based on the recent progress using cathepsin B for triggered drug release in tumors, cathepsin B-sensitive Gem prodrug-loaded CuNCs will be synthesized. Through CCR2 targeting mediated intracellular delivery into lysosomal of CCR2+ cells, Gem will be rapidly released following the cleavage of a cathepsin B-sensitive linker. As described in the above examples, the tumor inhibition was due to TA-PEG-Gem following the dissolution of CuNCs within TME and tumor cells. Because the IC50 of Gem is 2.5 times better than that of TA-PEG-Gem, this optimized design of cathepsin B-cleavable CuNCs-ECL1 i-GEM can significantly improve the PDAC treatment efficiency.

1. A CCR2-targeting composition comprising:
a CCR2 binding peptide comprising a TFLK sequence;
a chemotherapeutic agent moiety; and
a nanoparticle;
wherein,
the CCR2-targeting composition is capable of binding to CCR2;
the CCR2 binding peptide is no more than 200 amino acids in length;
the CCR2 binding peptide is conjugated to the nanoparticle by direct covalent bonding or with a linker; and
the chemotherapeutic agent moiety is conjugated to the nanoparticle by direct covalent bonding or with a linker.

2. (canceled)

3. The CCR2-targeting composition of claim 1, wherein the CCR2-targeting composition has a diameter less than about 10 nm.

4. The CCR2-targeting composition of claim 1, wherein the nanoparticle is an ultrasmall copper nanocluster.

5. The CCR2-targeting composition of claim 1, wherein the CCR2-targeting composition enables imaging of a tumor or delivery of a therapy to a tumor.

6. The CCR2-targeting composition of claim 4, wherein the chemotherapeutic agent moiety is gemcitabine, and the CCR2 binding peptide is ECL1i.

7. The CCR2-targeting composition of claim 1, wherein the linker is PEG, a PEG derivative, or a protease cleavable linker.

8. The CCR2-targeting composition of claim 7, wherein the linker is a cathepsin B-sensitive dipeptide linker.

9. A pharmaceutical composition comprising:
the CCR2-targeting composition of claim 1; and
an immunotherapeutic agent or a second chemotherapeutic agent moiety.

10. (canceled)

11. The pharmaceutical composition of claim 9, wherein the immunotherapeutic agent comprises at least one immune checkpoint inhibitor.

12. The pharmaceutical composition of claim 11, wherein the at least one immune checkpoint inhibitor is anti-CTLA antibodies, anti-PD-1 antibodies, or both.

13. The pharmaceutical composition of claim 9, wherein the second chemotherapeutic agent moiety is paclitaxel or nab-paclitaxel.

14. (canceled)

15. A method of treating a subject having cancer comprising: administering the CCR2-targeting composition of any claim 1.

16. The method of claim 15, further comprising administering an immunotherapy.

17. (canceled)

18. (canceled)

19. The method of claim 15, wherein the CCR2-targeting composition is Cu- or ⁶⁴Cu-CuNPs-ECL1i-Gem.

20. The method of claim 15, wherein administration of the CCR2-targeting composition results in: elimination of CCR2+ myeloid cells, inhibition of tumor-associated macrophage recruitment by targeting CCR2, tumor necrosis, effective inhibition of tumor growth, or at least about 20% prolonged survival.

21-23. (canceled)

24. The method of claim 16, wherein the immunotherapy is administered before, after, or simultaneously with the CCR2-targeting composition.

25. The method of claim 15, wherein the CCR2-targeting composition comprises a biodegradable copper nanocluster (CuNC), wherein biodegradation of the CuNC enables release of the chemotherapeutic agent moiety within a tumor microenvironment (TME) to kill CCR2+ stromal cells including tumor-infiltrating inflammatory monocytes (IMs) and macrophages (TAMs) to change the TME to immunoresponsive.

26. (canceled)

27. The method of claim 15, wherein the cancer is pancreatic ductal adenocarcinoma (PDAC).

28. The method of claim 19, further comprising PET imaging of the subject following administration of the CCR2-targeting composition.

29. The method of claim 16, wherein the immunotherapy comprises an anti-CTLA4 checkpoint blockade immunotherapy, an anti-PD-1 checkpoint blockade immunotherapy, or both.
